(19) United States
(12) Patent Application Publication
(10) Pub. No.: US 2024/0185352 A1
(43) Pub. Date: Jun. 6, 2024

(54) RISK SCORE EVALUATION USING AGENT-BASED SIMULATIONS

(71) Applicant: Optum Services (Ireland) Limited, Dublin (IE)

(72) Inventor: Neill Michael Byrne, Dublin (IE)

(21) Appl. No.: 18/164,112

(22) Filed: Feb. 3, 2023

Related U.S. Application Data
(60) Provisional application No. 63/386,215, filed on Dec. 6, 2022.

(51) Int. Cl. G06Q 40/08

(52) U.S. Cl. CPC G06Q 40/08

(57) ABSTRACT

Various embodiments of the present disclosure describe simulation, prediction, and data augmentation techniques for intelligently evaluating predictive risk scores and/or one or more combinations thereof. The techniques include generating, using a risk prediction model, predictive risk scores for an agent dataset. The techniques include generating simulated risk scores for the agent dataset based on the predictive risk scores and a first performance metric for the risk prediction model. The techniques include generating refined risk scores for the agent dataset based on the simulated risk scores and a second performance metric for a target risk refinement model. The techniques include generating return metrics for the target risk refinement model based on one or more iterations of an agent-based simulation enabled by the predictive risk scores, simulated risk scores, and refined risk scores.

### CROSS-REFERENCE TO RELATED APPLICATIONS

This application claims the benefit of U.S. Provisional Application No. 63/386,215, entitled “DETERMINING BENEFITS OF COMBINED POLYGENIC RISK SCORES WITH CLINICAL RISK SCORES USING HEALTHCARE POLICY AGENT BASED SIMULATION,” and filed Dec. 6, 2022, the entire contents of which are hereby incorporated by reference.

### BACKGROUND

Various embodiments of the present disclosure address technical challenges related to the evaluation and selection of various risk score modeling techniques. Existing risk scoring techniques may be tailored to specific attributes for an input and may offer limited accuracy and performance depending on the characteristics of the specific attributes considered. At times, multiple risk scoring techniques may be combined to improve risk prediction; however, doing so increases costs (e.g., computing resources, timing costs, etc.) associated with the risk predictions without a clear understanding of the actual increases in accuracy and performance. Such insights regarding the trade-offs between using different combinations of risk scoring techniques may rely on datasets that may not be available. Lack of data and sufficient modeling techniques to overcome sparse datasets prevent the informed evaluation and selection of risk scoring techniques for certain domains. Various embodiments of the present disclosure make important contributions to various existing risk score modeling techniques by addressing these technical challenges.

### BRIEF SUMMARY

Various embodiments of the present disclosure provide simulation, prediction, and data augmentation techniques for evaluating particular combinations of predictive risk scoring techniques. The various embodiments of the present disclosure leverage statistical modeling to generate realistic synthetic risk scores for data entities of an evaluation domain. Unlike conventional evaluation techniques, the synthetic risk scores may be generated for a variety of risk scoring techniques without specific real-world datasets tailored to each technique targeted for evaluation. For instance, various embodiments of the present disclosure provide for the generation of simulated risk scores and refined risk scores that realistically simulate the use of a targeted risk scoring technique as well as ground truths for evaluating the technique. By doing so, various embodiments of the present disclosure enable improved agent-based simulation techniques for simulating events based on limited historical datasets.

In some embodiments, a computer-implemented method includes generating, by one or more processors and using a risk prediction model, a plurality of predictive risk scores for an agent dataset associated with an agent-based simulation; generating, by the one or more processors, a plurality of simulated risk scores for the agent dataset based on the plurality of predictive risk scores and a first performance metric corresponding to the risk prediction model; generating, by the one or more processors, a plurality of refined risk scores for the agent dataset based on the plurality of simulated risk scores and a second performance metric corresponding to a target risk refinement model; and generating, by the one or more processors, one or more return metrics for the target risk refinement model based on one or more iterations of the agent-based simulation, wherein an iteration of the agent-based simulation is performed using the plurality of predictive risk scores, the plurality of simulated risk scores, and the plurality of refined risk scores.

In some embodiments, a computing apparatus includes a memory and one or more processors communicatively coupled to the memory. The one or more processors may be configured to: generate, using a risk prediction model, a plurality of predictive risk scores for an agent dataset associated with an agent-based simulation; generate a plurality of simulated risk scores for the agent dataset based on the plurality of predictive risk scores and a first performance metric corresponding to the risk prediction model; generate a plurality of refined risk scores for the agent dataset based on the plurality of simulated risk scores and a second performance metric corresponding to a target risk refinement model; and generate one or more return metrics for the target risk refinement model based on one or more iterations of the agent-based simulation, wherein an iteration of the agent-based simulation is performed using the plurality of predictive risk scores, the plurality of simulated risk scores, and the plurality of refined risk scores.

In some embodiments, one or more non-transitory computer-readable storage media includes instructions that, when executed by one or more processors, cause the one or more processors to: generate, using a risk prediction model, a plurality of predictive risk scores for an agent dataset associated with an agent-based simulation; generate a plurality of simulated risk scores for the agent dataset based on the plurality of predictive risk scores and a first performance metric corresponding to the risk prediction model; generate a plurality of refined risk scores for the agent dataset based on the plurality of simulated risk scores and a second performance metric corresponding to a target risk refinement model; and generate one or more return metrics for the target risk refinement model based on one or more iterations of the agent-based simulation, wherein an iteration of the agent-based simulation is performed using the plurality of predictive risk scores, the plurality of simulated risk scores, and the plurality of refined risk scores.

### BRIEF DESCRIPTION OF THE DRAWINGS

FIG. 1 illustrates an example computing system in accordance with one or more embodiments of the present disclosure.

FIG. 2 is a schematic diagram showing a system computing architecture in accordance with some embodiments discussed herein.

FIG. 3 is a dataflow diagram showing example data structures for evaluating and selecting one or more risk scoring techniques in accordance with some embodiments discussed herein.

FIG. 4 is an operational example of AUC-based simulated risk scores that are generated based on an AUC metric for a risk prediction model in accordance with some embodiments discussed herein.

FIG. 5 is an operational example of R2-based simulated risk scores that are generated based on an R2 metric for the risk prediction model in accordance with some embodiments discussed herein.

FIG. 6 is an operational example for generating refined risk scores in accordance with some embodiments discussed herein.

FIG. 7 is a flowchart showing an example of a process for generating risk scores for an agent-based simulation in accordance with some embodiments discussed herein.

FIG. 8 is a flowchart showing an example process for performing an iteration of an agent-based simulation in accordance with some embodiments discussed herein.

FIG. 9 is a flowchart showing an example of a process for performing an agent-based simulation in accordance with some embodiments discussed herein.

### DETAILED DESCRIPTION

Various embodiments of the present disclosure are described more fully hereinafter with reference to the accompanying drawings, in which some, but not all embodiments of the present disclosure are shown. Indeed, the present disclosure may be embodied in many different forms and should not be construed as limited to the embodiments set forth herein; rather, these embodiments are provided so that the present disclosure will satisfy applicable legal requirements. The term “or” is used herein in both the alternative and conjunctive sense, unless otherwise indicated. The terms “illustrative” and “example” are used to be examples with no indication of quality level. Terms such as “computing,” “determining,” “generating,” and/or similar words are used herein interchangeably to refer to the creation, modification, or identification of data. Further, “based on,” “based at least in part on,” “based at least on,” “based upon,” and/or similar words are used herein interchangeably in an open-ended manner such that they do not necessarily indicate being based only on or based solely on the referenced element or elements unless so indicated. Like numbers refer to like elements throughout.

### I. Computer Program Products, Methods, and Computing Entities

Embodiments of the present disclosure may be implemented in various ways, including as computer program products that comprise articles of manufacture. Such computer program products may include one or more software components including, for example, software objects, methods, data structures, or the like. A software component may be coded in any of a variety of programming languages. An illustrative programming language may be a lower-level programming language such as an assembly language associated with a particular hardware architecture and/or operating system platform. A software component comprising assembly language instructions may require conversion into executable machine code by an assembler prior to execution by the hardware architecture and/or platform. Another example programming language may be a higher-level programming language that may be portable across multiple architectures. A software component comprising higher-level programming language instructions may require conversion to an intermediate representation by an interpreter or a compiler prior to execution.

Other examples of programming languages include, but are not limited to, a macro language, a shell or command language, a job control language, a script language, a database query or search language, and/or a report writing language. In one or more example embodiments, a software component comprising instructions in one of the foregoing examples of programming languages may be executed directly by an operating system or other software component without having to be first transformed into another form. A software component may be stored as a file or other data storage construct. Software components of a similar type or functionally related may be stored together, such as in a particular directory, folder, or library. Software components may be static (e.g., pre-established or fixed) or dynamic (e.g., created or modified at the time of execution).

A computer program product may include a non-transitory computer-readable storage medium storing applications, programs, program modules, scripts, source code, program code, object code, byte code, compiled code, interpreted code, machine code, executable instructions, and/or the like (also referred to herein as executable instructions, instructions for execution, computer program products, program code, and/or similar terms used herein interchangeably). Such non-transitory computer-readable storage media include all computer-readable media (including volatile and non-volatile media).

In some embodiments, a non-volatile computer-readable storage medium may include a floppy disk, flexible disk, hard disk, solid-state storage (SSS) (e.g., a solid state drive (SSD), solid state card (SSC), solid state module (SSM), enterprise flash drive, magnetic tape, or any other non-transitory magnetic medium, and/or the like. A non-volatile computer-readable storage medium may also include a punch card, paper tape, optical mark sheet (or any other physical medium with patterns of holes or other optically recognizable indicia), compact disc readonly memory (CD-ROM), compact disc-rewritable (CD-RW), digital versatile disc (DVD), Blu-ray disc (BD), any other non-transitory optical medium, and/or the like. Such a non-volatile computer-readable storage medium may also include read-only memory (ROM), programmable read-only memory (PROM), erasable programmable read-only memory (EPROM), electrically erasable programmable read-only memory (EEPROM), flash memory (e.g., Serial, NAND, NOR, and/or the like), multimedia memory cards (MMC), secure digital (SD) memory cards, SmartMedia cards, CompactFlash (CF) cards, Memory Sticks, and/or the like. Further, a non-volatile computer-readable storage medium may also include conductive-bridging random-access memory (CBRAM), phase-change random-access memory (PRAM), ferroelectric random-access memory (FeRAM), non-volatile random-access memory (NVRAM), magnetoresistive random-access memory (MRAM), resistive random-access memory (RRAM), Silicon-Oxide-Nitride-Oxide-Silicon memory (SONOS), floating junction gate random-access memory (FJG RAM), Millipede memory, racetrack memory, and/or the like.

In some embodiments, a volatile computer-readable storage medium may include random-access memory (RAM), dynamic random-access memory (DRAM), static random-access memory (SRAM), fast page mode dynamic random-access memory (FPM DRAM), extended data-out dynamic random-access memory (EDO DRAM), synchronous dynamic random-access memory (SDRAM), double data rate synchronous dynamic random-access memory (DDR SDRAM), double data rate type two synchronous dynamic random-access memory (DDR2 SDRAM), double data rate type three synchronous dynamic random-access memory (DDR3 SDRAM), Rambus dynamic random-access memory (RDRAM), Twin Transistor RAM (TTRAM), Thyristor RAM (T-RAM), Zero-capacitor (Z-RAM), Rambus in-line memory module (RIMM), dual in-line memory module (DIMM), single in-line memory module (SIMM), video random-access memory (VRAM), cache memory (including various levels), flash memory, register memory, and/or the like. It will be appreciated that where embodiments are described to use a computer-readable storage medium, other types of computer-readable storage media may be substituted for or used in addition to the computer-readable storage media described above.

As should be appreciated, various embodiments of the present disclosure may also be implemented as methods, apparatus, systems, computing devices, computing entities, and/or the like. As such, embodiments of the present disclosure may take the form of an apparatus, system, computing device, computing entity, and/or the like executing instructions stored on a computer-readable storage medium to perform certain steps or operations. Thus, embodiments of the present disclosure may also take the form of an entirely hardware embodiment, an entirely computer program product embodiment, and/or an embodiment that comprises combination of computer program products and hardware performing certain steps or operations.

Embodiments of the present disclosure are described below with reference to block diagrams and flowchart illustrations. Thus, it should be understood that each block of the block diagrams and flowchart illustrations may be implemented in the form of a computer program product, an entirely hardware embodiment, a combination of hardware and computer program products, and/or apparatus, systems, computing devices, computing entities, and/or the like carrying out instructions, operations, steps, and similar words used interchangeably (e.g., the executable instructions, instructions for execution, program code, and/or the like) on a computer-readable storage medium for execution. For example, retrieval, loading, and execution of code may be performed sequentially such that one instruction is retrieved, loaded, and executed at a time. In some example embodiments, retrieval, loading, and/or execution may be performed in parallel such that multiple instructions are retrieved, loaded, and/or executed together. Thus, such embodiments may produce specially configured machines performing the steps or operations specified in the block diagrams and flowchart illustrations. Accordingly, the block diagrams and flowchart illustrations support various combinations of embodiments for performing the specified instructions, operations, or steps.

### II. Example Framework

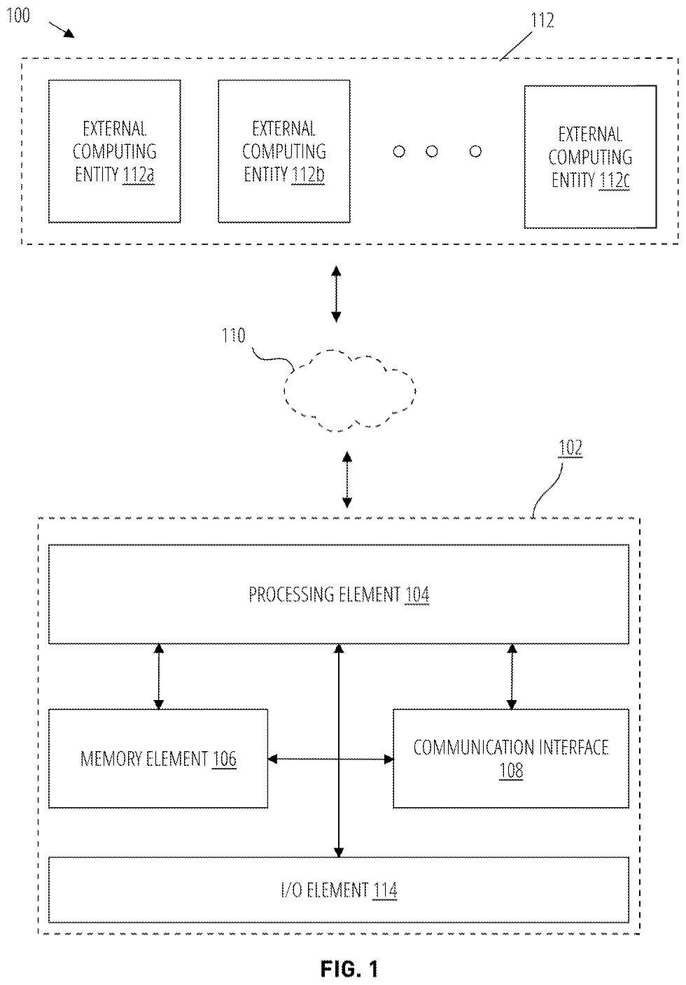

FIG. 1 illustrates an example computing system 100 in accordance with one or more embodiments of the present disclosure. The computing system 100 may include a predictive computing entity 102 and/or one or more external computing entities 112a-c communicatively coupled to the predictive computing entity 102 using one or more wired and/or wireless communication techniques. The predictive computing entity 102 may be specially configured to perform one or more steps/operations of one or more techniques described herein. In some embodiments, the predictive computing entity 102 may include and/or be in association with one or more mobile device(s), desktop computer(s), laptop(s), server(s), cloud computing platform(s), and/or the like. In some example embodiments, the predictive computing entity 102 may be configured to receive and/or transmit one or more datasets, objects, and/or the like from and/or to the external computing entities 112a-c to perform one or more steps/operations of one or more techniques (e.g., evaluation techniques, simulation techniques, prediction techniques, data augmentation techniques, etc.) described herein.

The external computing entities 112a-c, for example, may include and/or be associated with one or more data centers, claim centers, health care providers, and/or any other external entity that may be configured to receive, store, and/or process portions of one or more real world and/or synthetic dataset. The data centers, for example, may be associated with one or more data repositories storing historical, synthetic, and/or real time entity data (e.g., medical records, demographic information, etc.) that may, in some circumstances, be processed by the predictive computing entity 102 to generate one or more agent data objects as described herein. In some embodiments, one or more of the external computing entities 112a-c may include one or more processing entities that leverage one or more simulation techniques (e.g., agent-based simulation techniques, etc.) to generate the one or more return metrics for one or more predictive risk scoring techniques. In such a case, the predictive computing entity 102 may be configured to evaluate the one or more predictive risk scoring techniques using one or more of the simulation techniques described herein.

The predictive computing entity 102 may include, or be in communication with, one or more processing elements 104 (also referred to as processors, processing circuitry, digital circuitry, and/or similar terms used herein interchangeably) that communicate with other elements within the predictive computing entity 102 via a bus, for example. As will be understood, the predictive computing entity 102 may be embodied in a number of different ways. The predictive computing entity 102 may be configured for a particular use or configured to execute instructions stored in volatile or non-volatile media or otherwise accessible to the processing element 104. As such, whether configured by hardware or computer program products, or by a combination thereof, the processing element 104 may be capable of performing steps or operations according to embodiments of the present disclosure when configured accordingly.

In one embodiment, the predictive computing entity 102 may further include, or be in communication with, one or more memory elements 106. The memory element 106 may be used to store at least portions of the databases, database instances, database management systems, data, applications, programs, program modules, scripts, source code, object code, byte code, compiled code, interpreted code, machine code, executable instructions, and/or the like being executed by, for example, the processing element 104. Thus, the databases, database instances, database management systems, data, applications, programs, program modules, scripts, source code, object code, byte code, compiled code, interpreted code, machine code, executable instructions, and/or the like may be used to control certain aspects of the operation of the predictive computing entity 102 with the assistance of the processing element 104.

As indicated, in one embodiment, the predictive computing entity 102 may also include one or more communication interfaces 108 for communicating with various computing entities, e.g., external computing entities 112a-c, such as by communicating data, content, information, and/or similar terms used herein interchangeably that may be transmitted, received, operated on, processed, displayed, stored, and/or the like.

The computing system 100 may include one or more input/output (I/O) element(s) 114 for communicating with one or more users. An I/O element 114, for example, may include one or more user interfaces for providing and/or receiving information from one or more users of the computing system 100. The I/O element 114 may include one or more tactile interfaces (e.g., keypads, touch screens, etc.), one or more audio interfaces (e.g., microphones, speakers, etc.), visual interfaces (e.g., display devices, etc.), and/or the like. The I/O element 114 may be configured to receive user input through one or more of the user interfaces from a user of the computing system 100 and provide data to a user through the user interfaces.

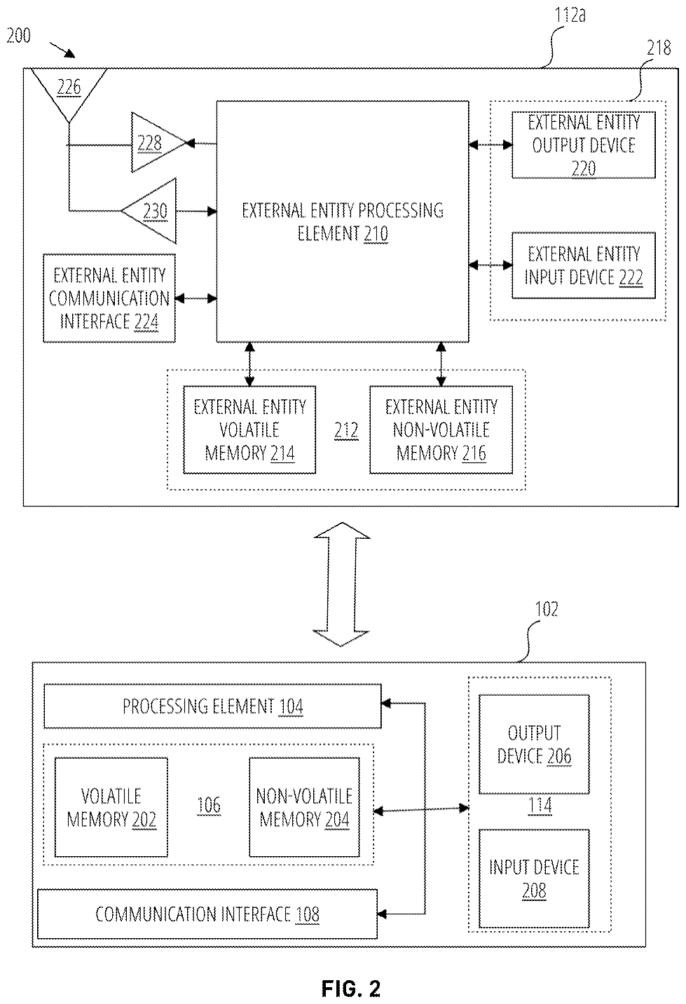

FIG. 2 is a schematic diagram showing a system computing architecture 200 in accordance with some embodiments discussed herein. In some embodiments, the system computing architecture 200 may include the predictive computing entity 102 and/or the external computing entity 112a of the computing system 100. The predictive computing entity 102 and/or the external computing entity 112a may include a computing apparatus, a computing device, and/or any form of computing entity configured to execute instructions stored on a computer-readable storage medium to perform certain steps or operations.

The predictive computing entity 102 may include a processing element 104, a memory element 106, a communication interface 108, and/or one or more I/O elements 114 that communicate within the predictive computing entity 102 via internal communication circuitry, such as a communication bus and/or the like.

The processing element 104 may be embodied as one or more complex programmable logic devices (CPLDs), microprocessors, multi-core processors, coprocessing entities, application-specific instruction-set processors (ASIPs), microcontrollers, and/or controllers. Further, the processing element 104 may be embodied as one or more other processing devices or circuitry including, for example, a processor, one or more processors, various processing devices and/or the like. The term circuitry may refer to an entirely hardware embodiment or a combination of hardware and computer program products. Thus, the processing element 104 may be embodied as integrated circuits, application specific integrated circuits (ASICs), field programmable gate arrays (FPGAs), programmable logic arrays (PLAs), hardware accelerators, digital circuitry, and/or the like.

The memory element 106 may include volatile memory 202 and/or non-volatile memory 204. The memory element 106, for example, may include volatile memory 202 (also referred to as volatile storage media, memory storage, memory circuitry and/or similar terms used herein interchangeably). In one embodiment, a volatile memory 202 may include random-access memory (RAM), dynamic random-access memory (DRAM), static random-access memory (SRAM), fast page mode dynamic random-access memory (FPM DRAM), extended data-out dynamic random-access memory (EDO DRAM), synchronous dynamic random-access memory (SDRAM), double data rate synchronous dynamic random-access memory (DDR SDRAM), double data rate type two synchronous dynamic random-access memory (DDR2 SDRAM), double data rate type three synchronous dynamic random-access memory (DDR3 SDRAM), Rambus dynamic random-access memory (RDRAM), Twin Transistor RAM (TTRAM), Thyristor RAM (T-RAM), Zero-capacitor (Z-RAM), Rambus in-line memory module (RIMM), dual in-line memory module (DIMM), single in-line memory module (SIMM), video random-access memory (VRAM), cache memory (including various levels), flash memory, register memory, and/or the like. It will be appreciated that where embodiments are described to use a computer-readable storage medium, other types of computer-readable storage media may be substituted for or used in addition to the computer-readable storage media described above.

The memory element 106 may include non-volatile memory 204 (also referred to as non-volatile storage, memory, memory storage, memory circuitry and/or similar terms used herein interchangeably). In one embodiment, the non-volatile memory 204 may include one or more non-volatile storage or memory media, including, but not limited to, hard disks, ROM, PROM, EPROM, EEPROM, flash memory, MMCs, SD memory cards, Memory Sticks, CBRAM, PRAM, FeRAM, NVRAM, MRAM, RRAM, SONOS, FJG RAM, Millipede memory, racetrack memory, and/or the like.

In one embodiment, a non-volatile memory 204 may include a floppy disk, flexible disk, hard disk, solid-state storage (SSS) (e.g., a solid-state drive (SSD)), solid state card (SSC), solid state module (SSM), enterprise flash drive, magnetic tape, or any other non-transitory magnetic medium, and/or the like. A non-volatile memory 204 may also include a punch card, paper tape, optical mark sheet (or any other physical medium with patterns of holes or other optically recognizable indicia), compact disc read-only memory (CD-ROM), compact disc-rewritable (CD-RW), digital versatile disc (DVD), Blu-ray disc (BD), any other non-transitory optical medium, and/or the like. Such a non-volatile memory 204 may also include read-only memory (ROM), programmable read-only memory (PROM), erasable programmable read-only memory (EPROM), electrically erasable programmable read-only memory (EEPROM), flash memory (e.g., Serial, NAND, NOR, and/or the like), multimedia memory cards (MMC), secure digital (SD) memory cards, SmartMedia cards, CompactFlash (CF) cards, Memory Sticks, and/or the like. Further, a non-volatile computer-readable storage medium may also include conductive-bridging random-access memory (CBRAM), phase-change random-access memory (PRAM), ferroelectric random-access memory (FeRAM), non-volatile random-access memory (NVRAM), magnetoresistive random-access memory (MRAM), resistive random-access memory (RRAM), Silicon-Oxide-Nitride-Oxide-Silicon memory (SONOS), floating junction gate random-access memory (FJG RAM), Millipede memory, racetrack memory, and/or the like.

As will be recognized, the non-volatile memory 204 may store databases, database instances, database management systems, data, applications, programs, program modules, scripts, source code, object code, byte code, compiled code, interpreted code, machine code, executable instructions, and/or the like. The term database, database instance, database management system, and/or similar terms used herein interchangeably may refer to a collection of records or data that is stored in a computer-readable storage medium using one or more database models, such as a hierarchical database model, network model, relational model, entity-relationship model, object model, document model, semantic model, graph model, and/or the like.

The memory element 106 may include a non-transitory computer-readable storage medium for implementing one or more embodiments of the present disclosure including as a computer-implemented method configured to perform one or more steps/operations described herein. For example, the non-transitory computer-readable storage medium may include instructions that when executed by a computer (e.g., processing element 104), cause the computer to perform one or more steps/operations of the present disclosure. For instance, the memory element 106 may store instructions that, when executed by the processing element 104, configure the predictive computing entity 102 to perform one or more step/operations described herein.

Embodiments of the present disclosure may be implemented in various ways, including as computer program products that comprise articles of manufacture. Such computer program products may include one or more software components including, for example, software objects, methods, data structures, or the like. A software component may be coded in any of a variety of programming languages. An illustrative programming language may be a lower-level programming language, such as an assembly language associated with a particular hardware framework and/or operating system platform. A software component comprising assembly language instructions may require conversion into executable machine code by an assembler prior to execution by the hardware framework and/or platform. Another example programming language may be a higher-level programming language that may be portable across multiple frameworks. A software component comprising higher-level programming language instructions may require conversion to an intermediate representation by an interpreter or a compiler prior to execution.

Other examples of programming languages include, but are not limited to, a macro language, a shell or command language, a job control language, a script language, a database query, or search language, and/or a report writing language. In one or more example embodiments, a software component comprising instructions in one of the foregoing examples of programming languages may be executed directly by an operating system or other software component without having to be first transformed into another form. A software component may be stored as a file or other data storage construct. Software components of a similar type or functionally related may be stored together, such as in a particular directory, folder, or library. Software components may be static (e.g., pre-established or fixed) or dynamic (e.g., created or modified at the time of execution).

The predictive computing entity 102 may be embodied by a computer program product include non-transitory computer-readable storage medium storing applications, programs, program modules, scripts, source code, program code, object code, byte code, compiled code, interpreted code, machine code, executable instructions, and/or the like (also referred to herein as executable instructions, instructions for execution, computer program products, program code, and/or similar terms used herein interchangeably). Such non-transitory computer-readable storage media include all computer-readable media such as the volatile memory 202 and/or the non-volatile memory 204.

The predictive computing entity 102 may include one or more I/O elements 114. The I/O elements 114 may include one or more output devices 206 and/or one or more input devices 208 for providing and/or receiving information with a user, respectively. The output devices 206 may include one or more sensory output devices, such as one or more tactile output devices (e.g., vibration devices, such as direct current motors, and/or the like), one or more visual output devices (e.g., liquid crystal displays, and/or the like), one or more audio output devices (e.g., speakers, and/or the like), and/or the like. The input devices 208 may include one or more sensory input devices, such as one or more tactile input devices (e.g., touch sensitive displays, push buttons, and/or the like), one or more audio input devices (e.g., microphones, and/or the like), and/or the like.

In addition, or alternatively, the predictive computing entity 102 may communicate, via a communication interface 108, with one or more external computing entities such as the external computing entity 112a. The communication interface 108 may be compatible with one or more wired and/or wireless communication protocols.

For example, such communication may be executed using a wired data transmission protocol, such as fiber distributed data interface (FDDI), digital subscriber line (DSL), Ethernet, asynchronous transfer mode (ATM), frame relay, data over cable service interface specification (DOCSIS), or any other wired transmission protocol. In addition, or alternatively, the predictive computing entity 102 may be configured to communicate via wireless external communication using any of a variety of protocols, such as general packet radio service (GPRS), Universal Mobile Telecommunications System (UMTS), Code Division Multiple Access 2000 (CDMA2000), CDMA2000 1× (1×RTT), Wideband Code Division Multiple Access (WCDMA), Global System for Mobile Communications (GSM), Enhanced Data rates for GSM Evolution (EDGE), Time Division-Synchronous Code Division Multiple Access (TD-SCDMA), Long Term Evolution (LTE), Evolved Universal Terrestrial Radio Access Network (E-UTRAN), Evolution-Data Optimized (EVDO), High Speed Packet Access (HSPA), High-Speed Downlink Packet Access (HSDPA), IEEE 802.9 (Wi-Fi), Wi-Fi Direct, 802.16 (WiMAX), ultra-wideband (UWB), infrared (IR) protocols, near field communication (NFC) protocols, Wibree, Bluetooth protocols, wireless universal serial bus (USB) protocols, and/or any other wireless protocol.

The external computing entity 112a may include an external entity processing element 210, an external entity memory element 212, an external entity communication interface 224, and/or one or more external entity I/O elements 218 that communicate within the external computing entity 112a via internal communication circuitry, such as a communication bus and/or the like.

The external entity processing element 210 may include one or more processing devices, processors, and/or any other device, circuitry, and/or the like described with reference to the processing element 104. The external entity memory element 212 may include one or more memory devices, media, and/or the like described with reference to the memory element 106. The external entity memory element 212, for example, may include at least one external entity volatile memory 214 and/or external entity non-volatile memory 216. The external entity communication interface 224 may include one or more wired and/or wireless communication interfaces as described with reference to communication interface 108.

In some embodiments, the external entity communication interface 224 may be supported by one or more radio circuitry. For instance, the external computing entity 112a may include an antenna 226, a transmitter 228 (e.g., radio), and/or a receiver 230 (e.g., radio).

Signals provided to and received from the transmitter 228 and the receiver 230, correspondingly, may include signaling information/data in accordance with air interface standards of applicable wireless systems. In this regard, the external computing entity 112a may be capable of operating with one or more air interface standards, communication protocols, modulation types, and access types. More particularly, the external computing entity 112a may operate in accordance with any of a number of wireless communication standards and protocols, such as those described above with regard to the predictive computing entity 102.

Via these communication standards and protocols, the external computing entity 112a may communicate with various other entities using means such as Unstructured Supplementary Service Data (USSD), Short Message Service (SMS), Multimedia Messaging Service (MMS), Dual-Tone Multi-Frequency Signaling (DTMF), and/or Subscriber Identity Module Dialer (SIM dialer). The external computing entity 112a may also download changes, add-ons, and updates, for instance, to its firmware, software (e.g., including executable instructions, applications, program modules), operating system, and/or the like.

According to one embodiment, the external computing entity 112a may include location determining embodiments, devices, modules, functionalities, and/or the like. For example, the external computing entity 112a may include outdoor positioning embodiments, such as a location module adapted to acquire, for example, latitude, longitude, altitude, geocode, course, direction, heading, speed, universal time (UTC), date, and/or various other information/data. In one embodiment, the location module may acquire data, such as ephemeris data, by identifying the number of satellites in view and the relative positions of those satellites (e.g., using global positioning systems (GPS)). The satellites may be a variety of different satellites, including Low Earth Orbit (LEO) satellite systems, Department of Defense (DOD) satellite systems, the European Union Galileo positioning systems, the Chinese Compass navigation systems, Indian Regional Navigational satellite systems, and/or the like. This data may be collected using a variety of coordinate systems, such as the Decimal Degrees (DD), Degrees, Minutes, Seconds (DMS), Universal Transverse Mercator (UTM), Universal Polar Stereographic (UPS) coordinate systems, and/or the like. Alternatively, the location information/data may be determined by triangulating a position of the external computing entity 112a in connection with a variety of other systems, including cellular towers, Wi-Fi access points, and/or the like. Similarly, the external computing entity 112a may include indoor positioning embodiments, such as a location module adapted to acquire, for example, latitude, longitude, altitude, geocode, course, direction, heading, speed, time, date, and/or various other information/data. Some of the indoor systems may use various position or location technologies including RFID tags, indoor beacons or transmitters, Wi-Fi access points, cellular towers, nearby computing devices (e.g., smartphones, laptops) and/or the like. For instance, such technologies may include the iBeacons, Gimbal proximity beacons, Bluetooth Low Energy (BLE) transmitters, NFC transmitters, and/or the like. These indoor positioning embodiments may be used in a variety of settings to determine the location of someone or something to within inches or centimeters.

The external entity I/O elements 218 may include one or more external entity output devices 220 and/or one or more external entity input devices 222 that may include one or more sensory devices described herein with reference to the I/O elements 114. In some embodiments, the external entity I/O element 218 may include a user interface (e.g., a display, speaker, and/or the like) and/or a user input interface (e.g., keypad, touch screen, microphone, and/or the like) that may be coupled to the external entity processing element 210.

For example, the user interface may be a user application, browser, and/or similar words used herein interchangeably executing on and/or accessible via the external computing entity 112a to interact with and/or cause the display, announcement, and/or the like of information/data to a user. The user input interface may include any of a number of input devices or interfaces allowing the external computing entity 112a to receive data including, as examples, a keypad (hard or soft), a touch display, voice/speech interfaces, motion interfaces, and/or any other input device. In embodiments including a keypad, the keypad may include (or cause display of) the conventional numeric (0-9) and related keys (#, *, and/or the like), and other keys used for operating the external computing entity 112a and may include a full set of 054642/591003 alphabetic keys or set of keys that may be activated to provide a full set of alphanumeric keys. In addition to providing input, the user input interface may be used, for example, to activate or deactivate certain functions, such as screen savers, sleep modes, and/or the like.

### III. Examples of Certain Terms

In some embodiments, the term “real-world dataset” refers to a data structure that represents a plurality of recorded attributes for a target evaluation domain. The type, format, and/or values of the recorded attributes may depend on the evaluation domain. In some examples, the evaluation domain may include particular predictions (e.g., risk scoring predictions, etc.) for a population of interest (e.g., a group of individuals, etc.). The population of interest may identify a plurality of entities for which one or more simulation techniques may be applied to generate one or more predictive insights.

For instance, the real-world dataset may include a plurality of recorded attributes for a plurality of entities that represent common attributes as well as a variance of those attributes among the entities. In some examples, the plurality of entities may include a population of individuals within a geographic area of interest. The real-world dataset may include a recorded dataset, such as a census dataset, a biobank, medical database, population surveys, and/or the like, that accurately represents various attributes, such as ethnicity, age, gender, blood pressure, rheumatoid arthritis, history of mental illness, etc., of a population of individuals. As one example, the real-world dataset may include a portion of a National Health and Nutrition Examination Survey (NHANES) dataset.

In some embodiments, the term “agent dataset” refers to a data structure that includes a plurality of agent data objects associated with an evaluation domain. The type, format, and parameters of each data object may be based on the evaluation domain. In some examples, the agent dataset may be based on the real-world dataset. For example, the agent dataset may include a plurality agent data objects that are based on the plurality of entities represented by the real-world dataset. In some embodiments, the agent dataset includes a plurality of agent data objects that serve as synthetic agents for agent-based simulations involving a population of interest.

In some examples, the plurality agent data objects of an agent dataset may be generated to reproduce attribute patterns (e.g., attribute proportions, such as demographic patterns, etc.) represented by the plurality of recorded attributes of a population of interest. To do so, subgroups may be identified amongst the population of interest across several attributes such as age group, gender, ethnicity, etc. The plurality of agent data objects may be generated, using sampling and replacement techniques, to align the agent dataset with the identified subgroups.

In some embodiments, the term “agent data object” refers to a data entity of an agent dataset. An agent data object may include a data structure including a plurality of agent attributes corresponding to an individual component of the agent data set. For example, in the event that the agent dataset corresponds to a population of individuals, an agent data object may include a plurality of agent attributes that correspond to an individual of the population of interest. In some examples, the agent data object may correspond to an individual represented by a real-world dataset. By way of example, the agent data object may include one or more synthetic attributes that are based on recorded attributes for a real-world individual.

In some embodiments, the agent attributes may include a plurality of agent markers. An agent marker, for example, may include an attribute that may be used to generate one or more predictive risk scores for the agent data object. The agent markers may depend on the evaluation domain. For example, the evaluation domain may include a risk assessment domain for a population of interest. In such a case, the agent markers may include clinical markers used to generate clinical risk scores. As an example, agent markers for a clinical risk score for cardiovascular disease may include common attributes such as ethnicity, age, gender, blood pressure, presence of rheumatoid arthritis, history of mental illness, and/or the like.

In some embodiments, the term “predictive risk score” refers to a data value that describes a predicted likelihood (e.g., a risk) of a target outcome for an entity. The predictive risk score may include any datatype, format, and/or value that evaluates a particular risk of an entity with respect to the target outcome. The predictive risk score may depend on the evaluation domain. For example, the predictive risk score may be predicted by a risk prediction model for a particular evaluation domain. In some examples, the evaluation domain may include a clinical domain and the risk prediction model may include a clinical risk calculator. For example, the predictive risk score may be a clinical risk score for a disease, such as cardiovascular disease (CVD). For example, the predictive risk score may be generated using a standardized clinical risk scoring (CRS) calculation for diseases, such as atherosclerotic cardiovascular disease (ASCVD). These clinical risk scores may be based on clinical pooled cohort studies which measure agent markers (e.g., phenotypes, etc.) that are understood to be markers for a morbidity, disease, and/or other conditions.

In some embodiments, the term “risk prediction model” refers to a data entity that describes parameters, hyper-parameters, and/or defined operations of a rules-based algorithm, machine learning model (e.g., model including at least one of one or more rule-based layers, one or more layers that depend on trained parameters, coefficients, and/or the like), and/or the like. The risk prediction model may be configured to generate a predictive risk score for an entity (e.g., as represented by an agent data object, etc.) based on markers (e.g., agent markers, etc.) corresponding to the entity. In some examples, the risk prediction model may include one or more clinical risk scoring techniques, such as an ASCVD calculator, QRISK algorithm, and/or the like.

In some embodiments, the term “simulated risk score” refers to a data value that describes an actual likelihood (e.g., risk) of a target outcome for an entity. The simulated risk score may include any datatype, format, and/or value that evaluates an actual risk of an entity with respect to the target outcome. As described herein, the simulated risk score may be realized during an agent-based simulation to simulate actual occurrences of a target outcome for an agent data object. The simulated risk score may depend on the evaluation domain. In some examples, the evaluation domain may include a clinical domain and the simulated risk score may be representative of a patient's actual risk for a disease, such as CVD. For example, the simulated risk score may include a synthetic value that represents the hypothetical true underlying risk of an entity having an event over some time period.

In some embodiments, the term “first performance metric” refers to a data value that describes a performance of the risk prediction model and/or one or more outputs thereof. The first performance metric may include any datatype, format, and/or value that evaluates the accuracy and/or other indicators of performance of the risk prediction model. The first performance metric may include any one or a combination of a plurality of performance metrics. In some embodiments, the first performance metric is an area under the ROC Curve (AUC) metric. For example, the first performance metric may describe the accuracy of the risk prediction model using an AUC or C-statistic between a plurality of historical predictive risk scores and recorded outcomes corresponding thereto. In addition, or alternatively, in some embodiments, the first performance metric is an r-squared (R2) metric.

In some embodiments, the term “score perturbations” refers to a data value that may be applied to a predictive risk score to generate a simulated risk score. The score perturbation, for example, may include a data value that may be added, subtracted, and/or otherwise applied to a predictive risk score to generate a simulated risk score that deviates from the predictive risk score.

In some embodiments, the term “first plurality of simulated risk scores” refers to a data value that describes a sampled simulated risk score. For example, the plurality of simulated risk scores may be generated across a plurality of iterations. At each iteration, a new set of simulated risk scores may be generated, evaluated, and, in the event that they satisfy one or more selection criteria, stored. The first plurality of simulated risk scores includes a set of simulated risk scores for a particular iteration of the plurality of iterations. The first plurality of simulated risk scores may be stored as the plurality of simulated risk scores in the event that they satisfy the one or more selection criteria.

In some embodiments, the term “performance evaluation model” refers to a data entity that describes parameters, hyper-parameters, and/or defined operations of a rules-based algorithm, machine learning model (e.g., model including at least one of one or more rule-based layers, one or more layers that depend on trained parameters, coefficients, and/or the like), and/or the like. The performance evaluation model may be configured to evaluate an accuracy between a plurality of predictive risk scores with respect to a first plurality of simulated risk scores. In some examples, the accuracy between the predictive risk scores and the simulated risk scores may be compared to one or more selection criteria to determine whether to use the first plurality of simulated risk scores for an agent-based simulation.

In some embodiments, the performance evaluation model is configured to generate a simulated performance metric. The simulated performance metric may be descriptive of a simulated accuracy of predictive risk scores with respect to simulated risk scores. In some examples, the simulated risk scores may be realized to determine binary outcomes corresponding to the predictive risk scores. The performance evaluation model may include a logistic regression model configured to measure an AUC of predicting the realized binary outcomes using the predictive and the simulated risk scores.

In some embodiments, the term “refined risk score” refers to a data value that describes a refined predicted likelihood (e.g., a risk) of a target outcome for an entity. The refined risk score may include any datatype, format, and/or value that refines a predictive risk score for an entity with respect to the target outcome. The refined risk score may depend on the evaluation domain. For example, the refined risk score may be predicted by a target risk refinement model for a particular evaluation domain. In some examples, the evaluation domain may include a clinical domain and the target risk refinement model may include a risk calculator that may be used to refine a clinical risk score for an entity based on contextual information. By way of example, the clinical risk score may be refined using a polygenic risk score (PRS) (e.g., genetic risk score, genome-wide score, etc.) that predicts an estimated impact of one or more genetic variants on an entity's phenotype, typically calculated as a weighted sum of trait-associated alleles. In some examples, the refined risk score may be a combined risk measure based on a CRS (e.g., a predictive risk score) and a PRS for an entity.

In some embodiments, the term “target risk refinement model” refers to a data entity that describes parameters, hyper-parameters, and/or defined operations of a rules-based algorithm, machine learning model (e.g., model including at least one of one or more rule-based layers, one or more layers that depend on trained parameters, coefficients, and/or the like), and/or the like. The target risk refinement model may be leveraged to generate a refined risk score for an entity. For instance, the target risk refinement model may be configured to generate a secondary risk score (e.g., PRS, etc.) that may be combined with a predictive risk score (e.g., CRS, etc.) to generate a refined risk score. By way of example, the target risk refinement model may include a polygenic risk scoring algorithm configured to generate a PRS for an entity. In some embodiments, the agent-based simulation techniques of the present disclosure are leveraged to evaluate projected returns of refined risk scores (e.g., combined clinical-polygenic risk scores) generated using the target risk refinement model compared to unrefined, predictive risk scores (e.g., clinical risk scores).

In some embodiments, the term “second performance metric” refers to a data value that describes a performance of the target risk refinement model and/or one or more outputs thereof. The second performance metric may include any datatype, format, and/or value that evaluates the accuracy and/or other indicators of performance of the target risk refinement model. The second performance metric may include any one or a combination of a plurality of performance metrics. In some embodiments, the second performance metric is a reported AUC metric. For example, the second performance metric may describe the accuracy of the target risk refinement model using an AUC or C-statistic between a plurality of historical refined risk scores and recorded outcomes corresponding thereto.

In some embodiments, the term “refined sample deviations” refers to a data value that may be applied to a predictive risk score to generate a refined risk score. The refined sample deviation, for example, may include a data value that may be added, subtracted, and/or otherwise applied to a predictive risk score to generate a refined risk score that deviates from the predictive risk score. The deviation caused by the refined sample deviation may represent a potential benefit of using a secondary risk score with the predictive risk score.

In some embodiments, the term “offset refined sample deviations” refers to a data value that may be applied to a predictive risk score to generate a refined risk score. The offset refined sample deviation may include a refined sample deviation from a standard normal distribution that is offset by an offset value (e.g., denoted as d). The offset value, for example, may include a Cohen's d that is determined based on the second performance metric. For example, the offset value may be defined as:

$$d = [ t - \frac{2.515517 + 0.802853 t + 0.0103328 {t}^{2}}{1 + 1.432788 t + 0.189269 {t}^{2} + 0.001308 {t}^{3}} ] * \sqrt{2}$$

$$where ,$$

$$t = \sqrt{ln ( \frac{1}{{( 1 - c )}^{2}}} .$$

In some embodiments, the term “simulated outcome” refers to a data entity that describes an outcome for an agent data object during one or more iterations of an agent-based simulation. The simulated outcome may include any datatype, format, and/or value that describes an event for an agent data object. The simulated outcome may depend on the evaluation domain. For example, in a clinical evaluation domain for CVD, the simulated outcome may represent a morbidity event (e.g., death), onset of disease symptoms, and/or other conditions for an agent data object.

In some embodiments, the term “adverse outcome” refers to one class of a simulated outcome. The adverse outcome may depend on the evaluation domain. As one example, in a clinical evaluation domain for CVD, an adverse outcome may represent the occurrence of a morbidity event (e.g., death), onset of disease symptoms, and/or other conditions for an agent data object.

In some embodiments, the term “positive outcome” refers to one class of a simulated outcome. The positive outcome may depend on the evaluation domain. As one example, in a clinical evaluation domain for CVD, a positive outcome may represent the non-occurrence of a morbidity event (e.g., death), onset of disease symptoms, and/or other conditions for an agent data object.

In some embodiments, the term “return metric” refers to a data entity is output from an agent-based simulation. The return metric may include any datatype, format, and/or value that describes one or more insights derived from the agent-based simulation. The return metric may depend on the evaluation domain. In some examples, in a clinical evaluation domain for CVD, the return metric describes one or more long-term gains in quality of life improvements and/or reductions in healthcare spend by using (and/or not using) refined risk scores.

### IV. Overview, Technical Improvements, and Technical Advantages

Embodiments of the present disclosure present simulation, prediction, and data augmentation techniques that enable improved agent-based simulations for evaluating combinations of predictive risk scores. Various embodiments of the present disclosure overcome technical challenges that prevent the evaluation of different combinations of risk scores by applying statistical modeling to generate synthetic risk scores that realistically simulate the different combinations of risk scores based on limited real-world data. In this way, various embodiments of the present disclosure enable the evaluation and intelligent selection of various combinations of risk scoring methodologies that may be tailored to an evaluation domain.

Various embodiments of the present disclosure may be applied to any evaluation domain in which multiple risk scoring methods may be applied. In such cases, the performance (e.g., predictive accuracy, etc.) of different combinations of risk scoring methods may be difficult to evaluate due to a lack of real-world data tailored to certain targeted combinations. To overcome these technical challenges, the various embodiments of the present disclosure provide for data augmentation techniques for generating complementary synthetic risk scores for simulating real world combinations of risk scoring methods. For instance, predictive risk scores generating using a first predictive risk scoring model may be leveraged to generate refined risk scores that simulate the combination of the predictive risk score with a secondary risk score for which real world data is limited. This enables the evaluation of any of a variety of combinations of risk scores through agent-based simulations despite the absence of robust real world data sets.

As one example, the various embodiments of the present disclosure may be applied in a clinical evaluation domain to evaluate various risk scoring metrics for predicting the likelihood of clinical diseases, such as cardiovascular disease. The various risk scoring metrics may include CRS and/or PRS predictions for which limited datasets may be available. Even if specific datasets are available, such datasets may still be deficient for failing to contain longitudinal data which may be required to capture target outcomes or endpoints and thus still may not be used to evaluate combined PRS/CRS techniques. Some organizations such as health insurers may have vast amounts of clinical and/or administrative claims data, but little or no overlapping genotype information. If they are contemplating procuring or advancing the use of polygenic risk scoring techniques in the healthcare system by genotyping members, it may be difficult to understand the return on investment from using combined PRS/CRS to aid clinical decision making without implementing costly trials and a significant upfront investment. Using the various embodiments of the present disclosure, an agent-based simulation may be constructed which may give a projected return (e.g., return metrics) on investment on both a quality-of-life basis and a medical cost avoidance basis by introducing PRS in combination with CRS to improve clinical decision-making.

Example technologically advantageous embodiments of the present disclosure include a risk scoring evaluation techniques that (i) generate synthetic ground truths (e.g., simulated risk scores, etc.) by (a) permuting predictive risk scores by perturbing the scores by an amount which estimates the predictive risk scores' reported AUC accuracy or (b) perturbing (whilst holding fixed principal moments) by flipping sampled values until a desired correlation is achieved which matches the reported R² accuracy of the predictive risk scores, (ii) generate synthetic refined risk scores by using realized binary values from the previously generated simulated risk scores and sampling from a standard normal distribution and a standard normal distribution shifted by Cohen's d to achieve a target reported secondary risk score's AUC, and/or (iii) perform an agent-based simulation that applies three different synthetic risk scores—predictive risk scores, simulated risk scores, and refined risk scores—to evaluate the performance of a combination of risk scoring techniques in an evaluation domain.

### V. Example System Operations

As indicated, various embodiments of the present disclosure make important technical contributions to predictive risk scoring techniques. In particular, systems and methods are disclosed herein that implement techniques configured to evaluation the relative performance of various combinations of predictive risk scoring techniques, such as machine learning risk scoring, and/or the like. In this way, targeted risk scoring models may be evaluated and intelligently selected to tailor risk scoring techniques to various different evaluation domains, among other improvements described herein.

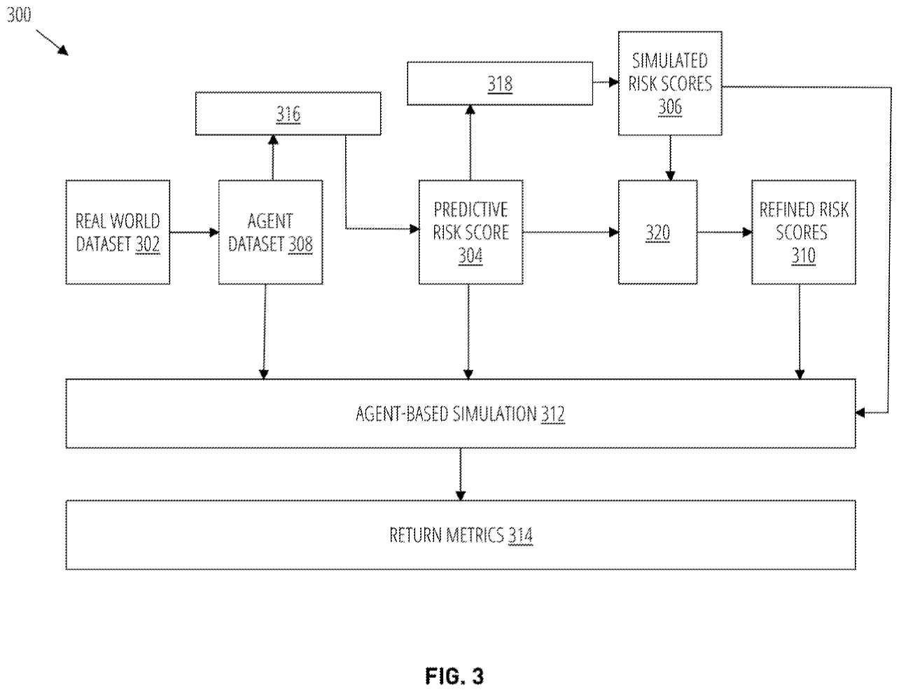

FIG. 3 is a dataflow diagram 300 showing example data structures for evaluating and selecting one or more risk scoring techniques in accordance with some embodiments discussed herein. The dataflow diagram 300 depicts a set of data structures and modules for realistically simulating one or more aspects of a real-world dataset 302 and/or one or more entities represented by the real-world dataset 302 to generate one or more return metrics 314 for a target risk refinement model 320. Each of the data structures and/or modules may be based on an evaluation domain for which the return metrics 314 are generated.

The return metrics 314 may be generated using agent-based simulation techniques that are grounded by real world information. The real-world information, for example, may be defined by the real-world dataset 302.

In some embodiments, the real-world dataset 302 is a data structure that represents a plurality of recorded attributes for a target evaluation domain. The type, format, and/or values of the recorded attributes may depend on the evaluation domain. In some examples, the evaluation domain may include particular predictions (e.g., risk scoring predictions, etc.) for a population of interest (e.g., a group of individuals, etc.). The population of interest may identify a plurality of entities for which one or more simulation techniques may be applied to generate one or more predictive insights.

For instance, the real-world dataset 302 may include a plurality of recorded attributes for a plurality of entities that represent common attributes as well as a variance of those attributes among the entities. In some examples, the plurality of entities may include a population of individuals within a geographic area of interest. The real-world dataset 302 may include a recorded dataset, such as a census dataset, a biobank, medical database, population surveys, and/or the like, that accurately represents various attributes, such as ethnicity, age, gender, blood pressure, rheumatoid arthritis, history of mental illness, etc., of a population of individuals. As one example, the real-world dataset 302 may include a portion of a National Health and Nutrition Examination Survey (NHANES) dataset.

In some embodiments, the real-world dataset 302 is received and/or leveraged to generate a synthetic data for use in performing an agent-based simulation 312. The synthetic data, for example, may be defined by an agent dataset 308. For instance, the agent dataset 308 may be generated based on the real-world dataset 302 for a population of interest. In some examples, the agent dataset 308 may include a plurality of agent data objects corresponding to the population of interest. Each agent data object of the plurality of agent data objects may include a plurality of agent markers derived from the population of interest.

In some embodiments, the agent dataset 308 is a data structure that includes a plurality of agent data objects associated with an evaluation domain. The type, format, and parameters of each data object may be based on the evaluation domain. In some examples, the agent dataset 308 may be based on the real-world dataset 302. For example, the agent dataset 308 may include a plurality agent data objects that is based on the plurality of entities represented by the real-world dataset 302. In some embodiments, the agent dataset includes a plurality of agent data objects that serve as synthetic agents for agent-based simulation 312 involving a population of interest.

In some examples, the plurality agent data objects of the agent dataset 308 may be generated to reproduce attribute patterns (e.g., attribute proportions, such as demographic patterns, etc.) represented by the plurality of recorded attributes of a population of interest. To do so, subgroups may be identified amongst the population of interest across several attributes, such as age group, gender, ethnicity, and/or the like. The plurality of agent data objects may be generated, using sampling and replacement techniques, to align the agent dataset 308 with the identified subgroups.

In some embodiments, an agent data object is a data entity of the agent dataset 308. An agent data object may include a data structure including a plurality of agent attributes corresponding to an individual component of the agent data set. For example, in the event that the agent dataset 308 corresponds to a population of individuals, an agent data object may include a plurality of agent attributes that correspond to an individual of the population of interest. In some examples, the agent data object may correspond to an individual represented by the real-world dataset 302. By way of example, the agent data object may include one or more synthetic attributes that are based on recorded attributes for a real-world individual.

In some embodiments, the agent attributes may include a plurality of agent markers. An agent marker, for example, may include an attribute that may be used to generate one or more predictive risk scores for the agent data object. The agent markers may depend on the evaluation domain. For example, the evaluation domain may include a risk assessment domain for the population of interest. In such a case, the agent markers may include clinical markers used to generate clinical risk scores. As an example, agent markers for a clinical risk score for cardiovascular disease may include common attributes such as ethnicity, age, gender, blood pressure, presence of rheumatoid arthritis, history of mental illness, and/or the like.

In some embodiments, the agent dataset 308 is received, generated, and/or leveraged to perform an agent-based simulation 312. The agent-based simulation 312, for example, may evaluate the performance of a target risk refinement model 320 by simulating one or more risk scores for the agent data objects of the agent dataset 308. For instance, a plurality of predictive risk scores 304 for the agent dataset 308 associated with the agent-based simulation 312 may be generated using the risk prediction model 316. For example, a predictive risk score for a particular agent data object may be generated based on a plurality of agent markers of the agent data object. The predictive risk score may represent a first predicted likelihood of an adverse outcome for the agent data object during the one or more iterations of the agent-based simulation 312.

In some embodiments, the predictive risk scores 304 are data values that describes predicted likelihoods (e.g., a risk) of a target outcome for entities represented by the plurality of agent data objects. The predictive risk score 304 may include any datatype, format, and/or value that evaluates a particular risk of an entity with respect to the target outcome. The predictive risk scores 304 may depend on the evaluation domain. For example, the predictive risk scores 304 may be predicted by a risk prediction model 316 for a particular evaluation domain. In some examples, the evaluation domain may include a clinical domain and the risk prediction model 316 may include a clinical risk calculator. For example, the predictive risk scores 304 may include clinical risk scores for a disease, such as CVD. For example, the predictive risk scores 304 may be generated using a standardized clinical risk scoring (CRS) calculation for diseases, such as atherosclerotic cardiovascular disease (ASCVD). These clinical risk scores may be based on clinical pooled cohort studies which measure agent markers (e.g., phenotypes, etc.) that are understood to be markers for a morbidity, disease, and/or other conditions.

In some embodiments, the risk prediction model 316 is a data entity that describes parameters, hyper-parameters, and/or defined operations of a rules-based algorithm, machine learning model (e.g., model including at least one of one or more rule-based layers, one or more layers that depend on trained parameters, coefficients, and/or the like), and/or the like. The risk prediction model 316 may be configured to generate the predictive risk scores 304 for entities (e.g., as represented by agent data objects, etc.) based on markers (e.g., agent markers, etc.) respectively corresponding to the entities. In some examples, the risk prediction model 316 may include one or more clinical risk scoring techniques, such as an ASCVD calculator, QRISK algorithm, and/or the like.

In some embodiments, the predictive risk scores 304 include a respective predictive risk score for each agent data object of the agent dataset 308. For instance, the risk prediction model 316 may be applied to each agent data object to generate a synthetic predictive risk score for the agent data object.

In some embodiments, the predictive risk scores 304 are normalized over a standard time horizon. For example, a risk prediction model 316 may be applied to each agent data object to generate a chosen predictive risk score (e.g., or multiple risk scores, e.g., ASCVD, QRISK3, etc.) for the agent dataset 308 (e.g., corresponding to a synthetic population of interest, etc.). In some examples, the predictive risk scores 304 may include an associated time-based risk score, such as a risk over ten years (e.g., a ten-year risk of developing diabetes, etc.), a risk over five years (e.g., a risk of having an adverse ASCVD event within 5 years, etc.).

The time-based risk scores may be normalized to a standard time horizon, such as a one-year time horizon, etc. To do so, the time-based risk scores may be converted from the original time horizon risk (e.g., ten-year, five-year, etc.) to a target time horizon risk (e.g., one year, etc.) using a Poisson unit rate transformation as follows:

$${x}_{j} = 1 - {e}^{( 1 + ln ( 1 - {x}_{i} ) \frac{{t}_{j}}{{t}_{i}} )} .$$

where, xⱼ may be the probability of an event occurring within the target time horizon, xᵢ may be the probability of the event occurring within the original time horizon and tᵢ and tⱼ may be the original and target lengths of the time horizons, respectively.

In some embodiments, the agent-based simulation 312 evaluates the performance of the predictive risk scores 304—and/or other risk scores as described herein—relative to actual, real world, risks for a plurality of entities. These actual, real world, risks may be represented by a plurality of simulated risk scores 306. For example, the simulated risk scores 306 may be generated for the agent dataset 308 based on the predictive risk score 304 and a first performance metric corresponding to the risk prediction model 316, as described herein. A simulated risk score for an agent data object may represent an actual likelihood of an adverse outcome for the agent data object during one or more iterations of the agent-based simulation 312.

In some embodiments, the simulated risk scores 306 are data values that respectively describe an actual likelihood (e.g., risk) of a target outcome for a plurality of entities. The simulated risk scores 306 may include any datatype, format, and/or value that evaluates an actual risk of an entity with respect to the target outcome. As described herein, the simulated risk scores 306 may be realized during an agent-based simulation 312 to simulate actual occurrences of a target outcome for an agent data object. The simulated risk scores 306 may depend on the evaluation domain. In some examples, the evaluation domain may include a clinical domain and the simulated risk scores 306 may be representative of a patient's actual risk for a disease, such as CVD. For example, the simulated risk scores 306 may include a synthetic value that represents the hypothetical true underlying risk of an entity having an event over some time period.

In some embodiments the simulated risk scores 306 are generated by permuting the predictive risk score 304 based on a first performance metric corresponding to the risk prediction model 316. The first performance metric may include a performance metric for the risk prediction model 316 and/or the predictive risk scores 304.

In some embodiments, the first performance metric is a data value that describes a performance of the risk prediction model 316 and/or one or more outputs thereof. The first performance metric may include any datatype, format, and/or value that evaluates the accuracy and/or other indicators of performance of the risk prediction model 316. The first performance metric may include any one or a combination of a plurality of performance metrics. In some embodiments, the first performance metric is an AUC metric. For example, the first performance metric may describe the accuracy of the risk prediction model 316 using an AUC or C-statistic between a plurality of historical predictive risk scores and recorded outcomes corresponding thereto. In addition, or alternatively, in some embodiments, the first performance metric is an R2 metric.

In some embodiments, the agent-based simulation 312 evaluates the performance of one or more combinations of risk scores related to the actual, real world, risks for a plurality of entities. For example, combinations of risk scores may be leveraged to refine each other in particular circumstances to generate refined risk scores 310. For instance, it may be possible to couple individually derived risk scores and combine them to create a risk score which has a greater accuracy than the individual measures alone. A technical challenge with using such scores is evaluating the potential combined risk measure accuracy in the absence of a dataset which has sufficient data (e.g., phenotype data for CRS, genotype data for PRS, etc.) to derive both risk scores.

Various embodiments of the simulation techniques of the present disclosure address these challenges by generating synthetic predictive risk scores 304, simulated risk scores 306, and refined risk scores 310 for use during an agent-based simulation 312. By way of example, some embodiments of the present disclosure utilize the simulated risk scores 306 (e.g., actual risk scores, etc.) to derive the refined risk scores 310 that hold both correlation with the predictive risk scores 304 and the reported accuracy thereof. For example, a plurality of refined risk scores 310 may be generated for the agent dataset 308 based on the plurality of simulated risk scores 306 and a second performance metric corresponding to a target risk refinement model 320. The refined risk scores 310 for each agent data object of the agent dataset 308 may represent a second predicted likelihood of an adverse outcome for an agent data object during the one or more iterations of the agent-based simulation 312.

In some embodiments, the refined risk scores 310 include data values that describe a refined predicted likelihood (e.g., a risk) of a target outcome for an entity. The refined risk scores 310 may include any datatype, format, and/or value that refines the predictive risk score 304 for a plurality of entities with respect to the target outcome. The refined risk scores 310 may depend on the evaluation domain. For example, the refined risk scores 310 may be predicted by a target risk refinement model 320 for a particular evaluation domain. In some examples, the evaluation domain may include a clinical domain and the target risk refinement model 320 may include a risk calculator that may be used to refine a clinical risk score for an entity based on contextual information. By way of example, the clinical risk score may be refined using a PRS (e.g., genetic risk score, genome-wide score, etc.) that predicts an estimated impact of one or more genetic variants on an entity's phenotype, typically calculated as a weighted sum of trait-associated alleles. In some examples, the refined risk scores 310 may be a combined risk measure based on a CRS (e.g., a predicted risk score) and a PRS for an entity.

In some embodiments, the target risk refinement model 320 is a data entity that describes parameters, hyper-parameters, and/or defined operations of a rules-based algorithm, machine learning model (e.g., model including at least one of one or more rule-based layers, one or more layers that depend on trained parameters, coefficients, and/or the like), and/or the like. The target risk refinement model 320 may be leveraged to generate refined risk scores 310 for a plurality of entities. For instance, the target risk refinement model 320 may be configured to generate a secondary risk score (e.g., PRS, etc.) that may be combined with a predictive risk score (e.g., CRS, etc.) to generate a refined risk score. By way of example, the target risk refinement model 320 may include a polygenic risk scoring algorithm configured to generate a PRS for an entity. In some embodiments, the agent-based simulation techniques of the present disclosure are leveraged to evaluate projected returns of the refined risk scores 310 (e.g., combined clinical-polygenic risk scores) generated using the target risk refinement model 320 compared to unrefined, predictive risk scores 304 (e.g., clinical risk scores).

In some embodiments, the second performance metric refers to a data value that describes a performance of the target risk refinement model 320 and/or one or more outputs thereof. The second performance metric may include any datatype, format, and/or value that evaluates the accuracy and/or other indicators of performance of the target risk refinement model 320. The second performance metric may include any one or a combination of a plurality of performance metrics. In some embodiments, the second performance metric is a reported AUC metric. For example, the second performance metric may describe the accuracy of the target risk refinement model 320 using an AUC or C-statistic between a plurality of historical refined risk scores and recorded outcomes corresponding thereto.

In some embodiments, a plurality of iterations of the agent-based simulation 312 are performed using the agent dataset 308, the predictive risk scores 304, the simulated risk scores 306, and/or the refined risk scores 310 to generate return metrics 314 for the target risk refinement model 320. For instance, the one or more return metrics 314 for the target risk refinement model 320 may be generated based one or more iterations of the agent-based simulation 312. An iteration of the agent-based simulation 312 may be performed using the plurality of predictive risk scores 304, the plurality of simulated risk scores 306, and the plurality of refined risk scores 310. The one or more return metrics 314 may be representative of a real-world benefit of using the target risk refinement model 320. By way of example, the agent-based simulation 312 (e.g., a healthcare policy agent-based simulation in a clinical evaluation domain, etc.) may illustrate the long-term benefits (e.g., gain in quality of life improvements, reduction in healthcare spend, etc. in a clinical evaluation domain, etc.) and/or downsides to using one or more different risk scoring techniques.

In some embodiments, the return metrics 314 include data entities that are output from an agent-based simulation 312. The return metrics 314 may include any datatype, format, and/or value that describes one or more insights derived from the agent-based simulation 312. The return metrics 314 may depend on the evaluation domain. In some examples, in a clinical evaluation domain for CVD, the return metrics 314 describe one or more long-term gains in quality of life improvements and/or reductions in healthcare spend by using (and/or not using) the refined risk scores 310.

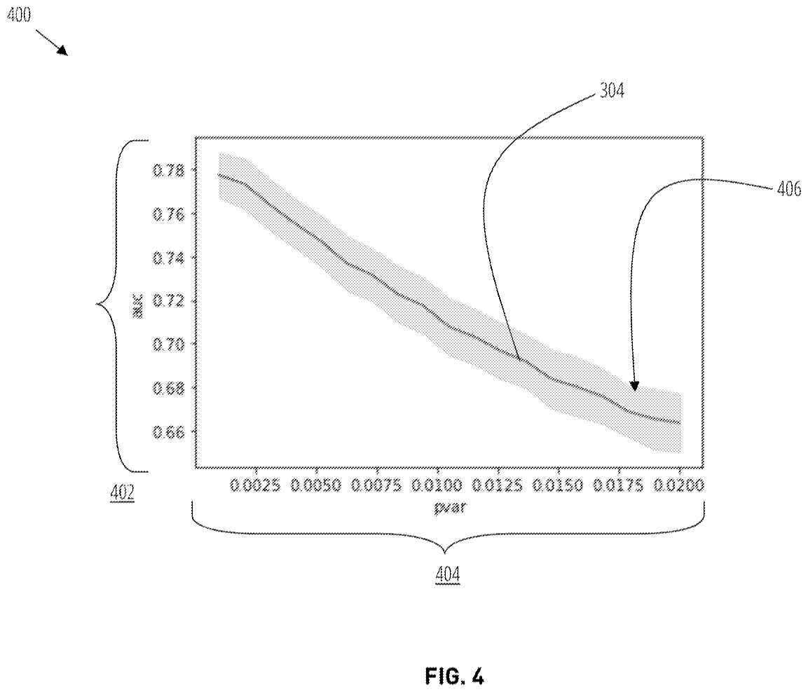

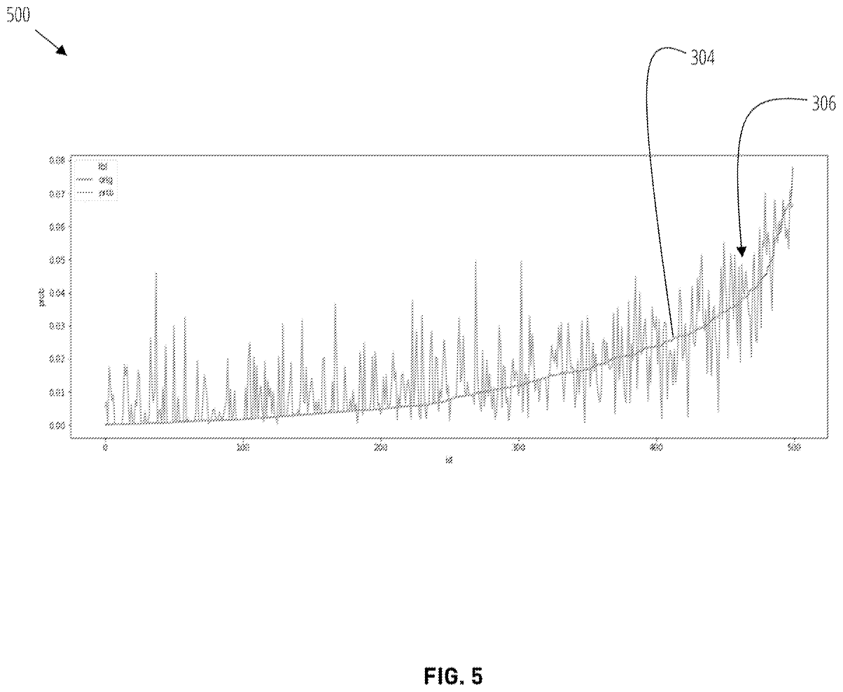

Operational examples for generating the simulated risk scores will now further be described with reference to FIGS. 4 and 5.

FIG. 4 is an operational example 400 of AUC-based simulated risk scores that are generated based on an AUC metric for a risk prediction model in accordance with some embodiments discussed herein. For example, in the operational example 400, the first performance metric 402 may include an AUC metric for the risk prediction model. The operational example 400 shows a plurality of score perturbations 406 for a plurality of predictive risk scores 304 as a function of the first performance metric 402 (e.g., AUC metric) and perturbation variances 404.

In some embodiments, the simulated risk scores are generated by perturbing the predictive risk score 304 (e.g., synthetic clinical risk score, etc.) to offset the scores such that the distribution remains unchanged while, if used as a predictor, the scores would have a detection/prediction accuracy equal to the first performance metric 402 (e.g., reported clinical risk scores AUC, etc.). The simulated risk scores may be generated by applying the score perturbations 406 to the predictive risk score 304 over a plurality perturbation iterations.

In some embodiments, the score perturbations 406 are generated for the plurality of predictive risk scores 304 over the plurality perturbation iterations. For instance, the score perturbations 406 may include individual increments (e.g., 0.01, etc.) for a range of perturbation variances 404 (e.g., 0.01 to 0.5, etc.). In some embodiments, the score perturbations 406 are data values that may be applied to the predictive risk scores 304 to generate simulated risk scores. The score perturbations 406, for example, may include a data value that may be added, subtracted, and/or otherwise applied to the predictive risk scores 304 to generate simulated risk scores that deviate from the predictive risk scores 304 to simulate the first performance metric 402.

In some embodiments, a first plurality of simulated risk scores is generated by augmenting one or more of the plurality of predictive risk scores 304 with the score perturbations 406. For example, samples (e.g., one for each agent data object of the agent dataset, etc.) may be generated from a random normal distribution with a standard deviation according to the value from the score perturbations 406. The value may be added from the sampled normal distribution to the predictive risk scores 304. If the resulting value is negative, the score may be resampled.

In some embodiments, the first plurality of simulated risk scores includes data values that describe sampled simulated risk scores for a particular perturbation iteration. For example, the plurality of simulated risk scores may be generated across a plurality of perturbation iterations. At each iteration, a new set of simulated risk scores may be generated, evaluated, and, in the event that they satisfy one or more selection criteria, stored. The first plurality of simulated risk scores includes a set of simulated risk scores for a particular iteration of the plurality of iterations. The first plurality of simulated risk scores may be stored as the plurality of simulated risk scores in the event that they satisfy the one or more selection criteria. The one or more selection criteria, for example, may be a detection/prediction accuracy defined by the first performance metric 402.

In some embodiments, a plurality of simulated outcomes is generated for the agent dataset based on the predictive risk scores 304. For example, the predictive risk scores 304 may be realized to a particular simulated outcome by sampling to binarize each score. For instance, if a predictive risk score is 0.25, then in 1 out of four iterations the score will be realized to a 1 (e.g., a first simulated outcome, etc.) and in 3 of the 4 iterations the score will be realized to a 0 (e.g., a second simulated outcome, etc.).

In some embodiments, a simulated performance metric may be generated for the risk prediction model by applying a performance evaluation model to the plurality of simulated outcomes. In some embodiments, the performance evaluation model includes to data entity that describes parameters, hyper-parameters, and/or defined operations of a rules-based algorithm, machine learning model (e.g., model including at least one of one or more rule-based layers, one or more layers that depend on trained parameters, coefficients, and/or the like), and/or the like. The performance evaluation model may be configured to evaluate an accuracy between a plurality of predictive risk scores 304 with respect to a first plurality of simulated risk scores. In some examples, the accuracy between the predictive risk scores 304 and the first plurality of simulated risk scores may be compared to one or more selection criteria to determine whether to use the first plurality of simulated risk scores for an agent-based simulation.

In some embodiments, the performance evaluation model is configured to generate a simulated performance metric. The simulated performance metric may be descriptive of a simulated accuracy of predictive risk scores 304 with respect to the first plurality of simulated risk scores. In some examples, the predictive risk score 304 may be realized to determine binary outcomes corresponding to the predictive risk scores 304. The performance evaluation model may include a logistic regression model configured to measure an AUC of predicting the realized binary outcomes using the first plurality of simulated risk scores.

In some embodiments, the plurality of simulated risk scores is generated based on a comparison between the simulated performance metric and the AUC metric. For example, the one or more selection criteria may define a performance threshold based on the AUC metric. The plurality of simulated risk scores may be generated based on the first plurality of simulated risk scores in the event that the simulated performance metric is within a threshold distance of the AUC metric. In the event that the simulated performance metric does not satisfy the selection criteria (e.g., the simulated performance metric is outside the threshold distance of the AUC metric), another perturbation iteration may be performed. These steps may be iteratively performed until a plurality of simulated risk scores are generated that satisfy the selection criteria.

FIG. 5 is an operational example 500 of R2-based simulated risk scores that are generated based on an R2 metric for the risk prediction model in accordance with some embodiments discussed herein. For example, in the operational example 500, the first performance metric may include an R2 metric for the risk prediction model. The operational example 500 shows a plurality of simulated risk scores 306 for a plurality of predictive risk scores 304. If an R2 metric is available, the simulated risk scores 306 may be generated by perturbing the predictive risk score 304 without realizing binary outcomes or using a performance evaluation model as described with reference to the operational example 400 of FIG. 4.

In some embodiments, an exponential distribution is generated based on an average predictive risk score of the plurality of predictive risk scores 304. For example, the exponential distribution may be generated with a mean given by the mean of the predictive risk scores 304. In some embodiments, a first plurality of simulated risk scores may be identified by sampling a plurality of distribution scores from the exponential distribution. By way of example, an equivalent number of samples from the exponential distribution may be samples as there are agent data objects of the agent dataset.

In some embodiments, the plurality of simulated risk scores is generated based on the plurality of predictive risk scores 304, the first plurality of simulated risk scores, and the R2 metric. For example, the square root of the R2 metric may be determined. The square root, for example, may include the target Pearson correlation. The predictive risk score 304 may be correlated with the sampled values whist maintaining distribution moments by: (i) flipping any two values from the first plurality of simulated risk scores, (ii) measuring the correlation between the predictive risk score 304 and the first plurality of simulated risk scores and, if the correlation moves closer to the correlation target, then keep the flipped values otherwise replace them to their original position. These steps may be continued until the correlation converges with one or more selection criteria (e.g., a minimum acceptable threshold of target correlation).

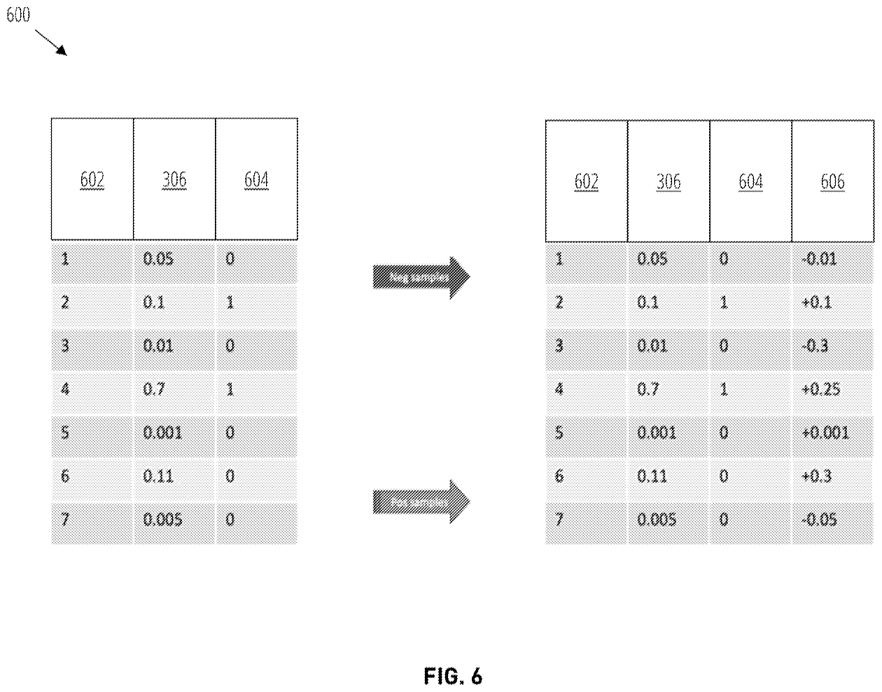

Operational examples for generating the refined risk scores will now further be described with reference to FIG. 6.

FIG. 6 is an operational example for generating refined risk scores in accordance with some embodiments discussed herein. A refined risk score may be generated for each of the agent data objects 602 of the agent dataset based on the predictive risk scores and the simulated risk scores 306 for the agent data objects 602. For example, the simulated risk scores 306 may be leveraged to generate a plurality of simulated outcomes 604 for the agent data objects 602. The simulated outcomes 604 may be leveraged to generate a plurality of sample deviations 606 for a plurality of predictive risk scores corresponding to the agent data objects 602. The sample deviations 606 may be applied to the predictive risk scores to generate a plurality of refined risk scores for the agent data objects 602.

In some embodiments, a Cohen's deviation is generated based on a second predictive metric for the target risk refinement model. For example, using the second predictive metric, a normal distribution may be created with the defined AUC using Cohen's deviation according to predefined constants. For example, Cohen's deviation may be derived by:

$$d = [ t - \frac{2.515517 + 0.802853 t + 0.0103328 {t}^{2}}{1 + 1.432788 t + 0.189269 {t}^{2} + 0.001308 {t}^{3}} ] * \sqrt{2}$$

$$where ,$$

$$t = \sqrt{ln ( \frac{1}{{( 1 - c )}^{2}}} .$$

In some embodiments, the plurality of simulated outcomes 604 for the agent dataset are generated based on the plurality of simulated risk scores 306. The plurality of simulated outcomes 604, for example, may identify at least one of a positive outcome (e.g., a 1, etc.) and/or an adverse outcome (e.g., a 0, etc.) for each of a plurality of agent data objects of the agent dataset.

In some embodiments, the simulated outcomes 604 include data entities that describe an outcome for an agent data object during one or more iterations of an agent-based simulation. The simulated outcome may include any datatype, format, and/or value that describes an event for the agent data objects 602. The simulated outcomes 604 may depend on the evaluation domain. For example, in a clinical evaluation domain for CVD, the simulated outcomes 604 may represent a morbidity event (e.g., death), onset of disease symptoms, and/or other conditions for the agent data objects 602.

In some embodiments, an adverse outcome may be one class of simulated outcomes 604. An adverse outcome may depend on the evaluation domain. As one example, in a clinical evaluation domain for CVD, an adverse outcome may represent the occurrence of a morbidity event (e.g., death), onset of disease symptoms, and/or other conditions for the agent data objects 602. In some embodiments, a positive outcome may be another class of simulated outcomes 604. A positive outcome may depend on the evaluation domain. As one example, in a clinical evaluation domain for CVD, a positive outcome may represent the non-occurrence of a morbidity event (e.g., death), onset of disease symptoms, and/or other conditions for the agent data objects 602.

In some embodiments, the simulated risk scores 306 may be realized by sampling the probability to give a binary occurrence value of positive or negative. In some examples, the agent data objects 602 may be sorted by the realized value to give a block of negative and a block of positives.

For example, an adverse subset of the plurality of agent data objects 602 may be identified from the agent dataset based on the plurality of simulated outcomes 604. Each adverse agent data object, for example, may be associated with an adverse outcome (e.g., 0, etc.). The adverse subset of the plurality of agent data objects 602 may be associated with a first subset of the plurality of predictive risk scores.

In addition, or alternatively, a positive subset of the plurality of agent data objects 602 may be identified from the agent dataset based on the plurality of simulated outcomes 604. Each positive agent data object of the positive subset, for example, may be associated with a positive outcome (e.g., 1, etc.). The positive subset of the plurality of agent data objects may be associated with a second subset of the plurality of predictive risk scores.

In some embodiments, the sample deviations 606 include at least one of one or more refined sample deviations and/or one or more offset refined sample deviations.

In some examples, the refined sample deviations may be identified from a standard normal distribution. For example, a standard normal distribution (e.g., with a mean of 0.0, standard deviation of 1.0, etc.) may be generated with a number of samples. The refined sample deviations may include data values that may be applied to the predictive risk scores to generate a refined risk score. The refined sample deviations, for example, may include a data value that may be added, subtracted, and/or otherwise applied to a predictive risk score to generate a refined risk score that deviates from the predictive risk score. The deviation caused by the refined sample deviation may represent a potential benefit of using a secondary risk score (e.g., a PRS, etc.) with the predictive risk score.

In some examples, the offset refined sample deviations may be identified from a standard normal distribution that is offset by the Cohen's deviation. For example, the offset refined sample deviations may include data values that may be applied to a predictive risk score to generate a refined risk score. The offset refined sample deviation may include a refined sample deviation from a standard normal distribution that is offset by an offset value (e.g., denoted as d). The offset value, for example, may include a Cohen's deviation that is determined based on the second performance metric as described herein.

In some embodiments, the plurality of refined risk scores is generated by randomly augmenting one or more of the plurality of predictive risk scores with at least one of the one or more refined sample deviations and/or the one or more offset refined sample deviations. For example, the standard normal distribution may be sampled to identify a refined sample deviation for each adverse agent data object. For instance, one or more of the first subset of the plurality of predictive risk scores may be randomly augmented with the one or more refined sample deviations to generate a first portion of the refined risk scores.

In some embodiments, the plurality of refined risk scores is generated by randomly augmenting one or more of the plurality of predictive risk scores with at least one of the one or more offset refined sample deviations. For example, the offset standard normal distribution may be sampled to identify an offset refined sample deviation for each positive agent data object. For instance, one or more of the second subset of the plurality of predictive risk scores may be randomly augmented with the one or more offset refined sample deviations to generate a second portion of the refined risk scores.

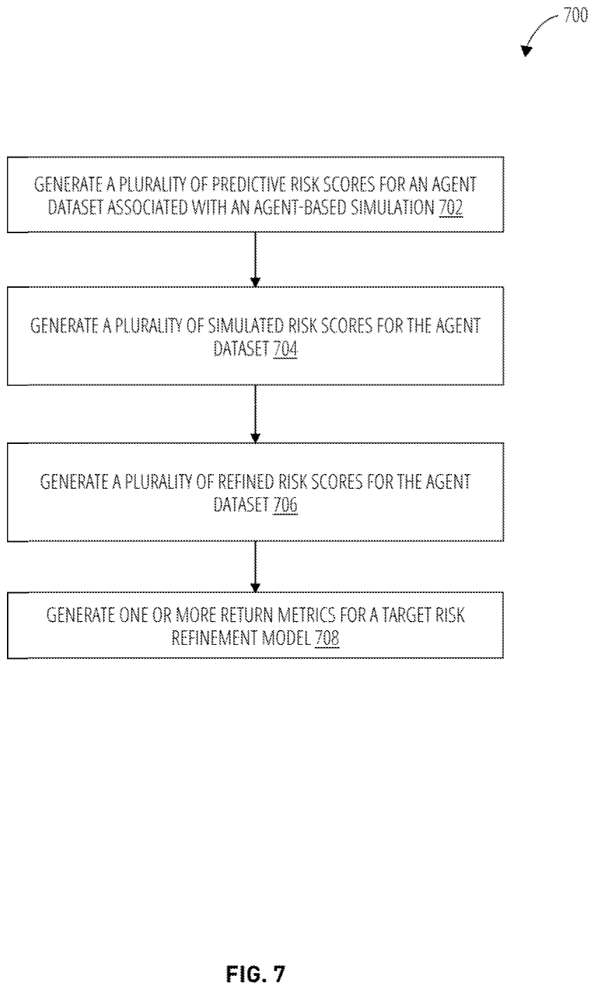

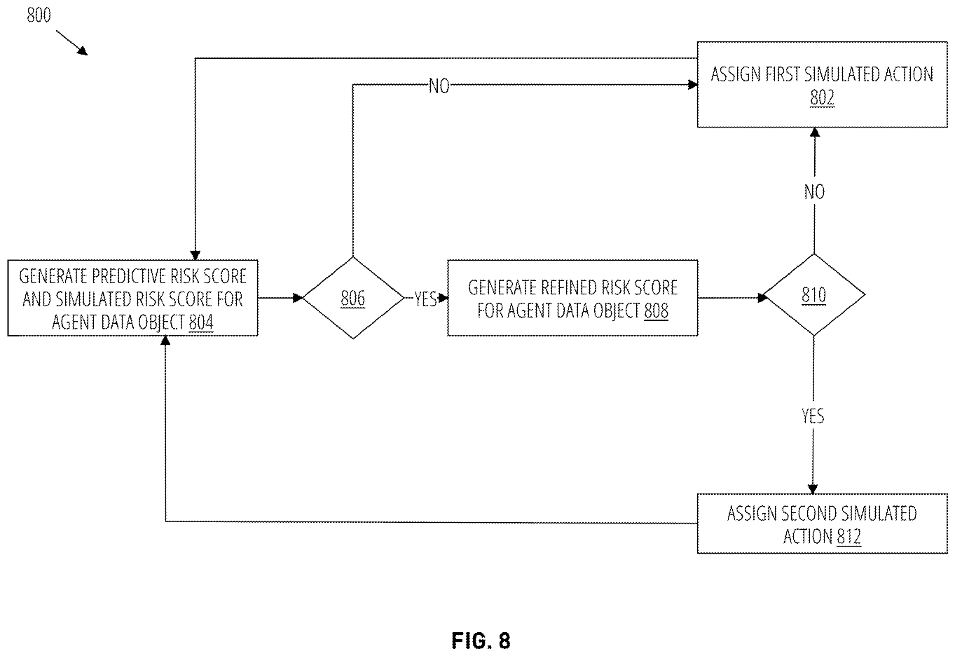

Example processes implementing one or more of the embodiments of the present disclosure will now further be described with reference to FIGS. 7 and 8.

FIG. 7 is a flowchart showing an example of a process 700 for generating risk scores for an agent-based simulation in accordance with some embodiments discussed herein. The process 700 leverages a plurality of different simulation, statistical, and data augmentation techniques to generate a plurality of synthetic risk scores for performing realistic agent-based simulations. The process 700 may be implemented by one or more computing devices, entities, and/or systems described herein. For example, via the various steps/operations of the process 700, the computing system 100 may leverage the simulation, statistical, and data augmentation techniques to overcome various limitations with conventional simulation and risk scoring techniques that are traditionally limited by the deficiencies of real-world data.

FIG. 7 illustrates an example process 700 for explanatory purposes. Although the example process 700 depicts a particular sequence of steps/operations, the sequence may be altered without departing from the scope of the present disclosure. For example, some of the steps/operations depicted may be performed in parallel or in a different sequence that does not materially impact the function of the process 700. In other examples, different components of an example device or system that implements the process 700 may perform functions at substantially the same time or in a specific sequence.

According to some examples, the process 700 includes, at step/operation 702, generating a plurality of predictive risk scores for an agent dataset associated with an agent-based simulation. For example, the computing system 100 may generate the plurality of predictive risk scores for the agent dataset associated with the agent-based simulation. For instance, the computing system 100 may generate, using a risk prediction model, the plurality of predictive risk scores for the agent dataset associated with the agent-based simulation.

In some embodiments, the computing system 100 generates the agent dataset based on a real-world dataset for a population of interest. The agent dataset may include a plurality of agent data objects corresponding to the population of interest. In some examples, an agent data object of the plurality of agent data objects includes a plurality of agent markers derived from the population of interest. In some embodiments, the computing system 100 generates, using the risk prediction model, a predictive risk score for the agent data object based on the plurality of agent markers. The predictive risk score for the agent data object may represent a first predicted likelihood of an adverse outcome for the agent data object during the one or more iterations of the agent-based simulation.

According to some examples, the process 700 includes, at step/operation 704, generating a plurality of simulated risk scores for the agent dataset. For example, the computing system 100 may generate the plurality of simulated risk scores for the agent dataset. For instance, the computing system 100 may generate the plurality of simulated risk scores for the agent dataset based on the plurality of predictive risk scores and a first performance metric corresponding to the risk prediction model. The simulated risk score for an agent data object may represent an actual likelihood of an adverse outcome for the agent data object during the one or more iterations of the agent-based simulation.

The first performance metric may include an AUC metric and/or an R2 metric for the risk prediction model. For an AUC metric, the computing system 100 may generate a plurality of score perturbations for the plurality of predictive risk scores, generate a first plurality of simulated risk scores by augmenting one or more of the plurality of predictive risk scores with the score perturbations, generate a plurality of simulated outcomes for the agent dataset based on the plurality of predictive risk scores, generate, using a performance evaluation model, a simulated performance metric for the risk prediction model based on the plurality of simulated outcomes, and generate the plurality of simulated risk scores based on a comparison between the simulated performance metric and the AUC metric. In addition, or alternatively, for an R2 metric, the computing system 100 may generate an exponential distribution based on an average predictive risk score of the plurality of predictive risk scores, identify a first plurality of simulated risk scores by sampling a plurality of distribution scores from the exponential distribution, and generate the plurality of simulated risk scores based on the plurality of predictive risk scores, the first plurality of simulated risk scores, and the R2 metric.

According to some examples, the process 700 includes, at step/operation 706, generating a plurality of refined risk scores for the agent dataset. For example, the computing system 100 may generate the plurality of refined risk scores for the agent dataset. For instance, the computing system 100 may generate the plurality of refined risk scores for the agent dataset based on the plurality of simulated risk scores and a second performance metric corresponding to a target risk refinement model. The refined risk score for the agent data object may represent a second predicted likelihood of an adverse outcome for the agent data object during the one or more iterations of the agent-based simulation.

In some embodiments, the computing system 100 generates a Cohen's deviation based on the second predictive metric for the target risk refinement model. The computing system 100 may identify one or more refined sample deviations from a standard normal distribution. In addition, or alternatively, the computing system 100 may identify one or more offset refined sample deviations from a standard normal distribution that is offset by the Cohen's deviation. The computing system 100 may generate the plurality of refined risk scores by randomly augmenting one or more of the plurality of predictive risk scores with at least one of the one or more refined sample deviations and/or the one or more offset refined sample deviations.

By way of example, the computing system 100 may generate a plurality of simulated outcomes for the agent dataset based on the plurality of simulated risk scores. The plurality of simulated outcomes may identify at least one of a positive outcome and/or an adverse outcome for each of a plurality of agent data objects of the agent dataset. The computing system 100 may identify an adverse subset of the plurality of agent data objects from the agent dataset based on the plurality of simulated outcomes. Each adverse agent data object of the adverse subset may be associated with the adverse outcome. The adverse subset of the plurality of agent data objects may be associated with a first subset of the plurality of predictive risk scores. The computing system 100 may randomly augment one or more of the first subset of the plurality of predictive risk scores with the one or more refined sample deviations.

In addition, or alternatively, the computing system 100 may identify a positive subset of the plurality of agent data objects from the agent dataset based on the plurality of simulated outcomes. Each positive agent data object of the positive subset may be associated with the positive outcome. The positive subset of the plurality of agent data objects may be associated with a second subset of the plurality of predictive risk scores. The computing system 100 may randomly augment one or more of the second subset of the plurality of predictive risk scores with the one or more offset refined sample deviations.

According to some examples, the process 700 includes, at step/operation 708, generating one or more return metrics for a target risk refinement model. For example, the computing system 100 may generate the one or more return metrics for a target risk refinement model. For instance, the computing system 100 may generate the one or more return metrics for the target risk refinement model based on one or more iterations of the agent-based simulation. Each iteration of the agent-based simulation may be performed using the plurality of predictive risk scores, the plurality of simulated risk scores, and/or the plurality of refined risk scores. The one or more return metrics are representative of a real-world benefit of using the target risk refinement model

FIG. 8 is a flowchart showing an example of a process 800 for performing an iteration of an agent-based simulation in accordance with some embodiments discussed herein. The process 800 leverages a plurality of different simulation, statistical, and data augmentation techniques to simulate the performance of a multiple different risk scoring techniques without relying on real world datasets for each of the targeted techniques. The process 800 may be implemented by one or more computing devices, entities, and/or systems described herein. For example, via the various steps/operations of the process 800, the computing system 100 may leverage the simulation, statistical, and data augmentation techniques to overcome various limitations with conventional simulation and risk scoring techniques that are traditionally limited by the deficiencies of real-world data.

The process 800 illustrates techniques for leveraging the three risk scores—predictive risk scores (e.g., CRS in clinical evaluation domain, etc.), simulated risk scores (e.g., actual risks, etc.), refined risk scores (e.g., CRS refined by PRS in clinical evaluation domain, etc.)—for an agent dataset matched to a real world dataset of the present disclosure to facilitate a full agent-based simulation configured to examine the impact of refining a first risk score with a second risk score in the real world. In a clinical evaluation domain, for example, the agent-based simulation may be applied to CRS and PRS predictions to evaluate whether the inclusion of costly PRS with CRS may lead to better health outcomes and/or reduced medical expenditure.

In some examples, an agent-based simulation may be performed by bifurcating an agent dataset into (i) agent data objects for which a refined risk score is generated (e.g., for which genotyping is performed) and (ii) agent data object for which a refined risk score is not generated (e.g., for which genotyping is not performed). In the event that the refined risk score enhances the predictive risk score by giving a more accurate risk prediction, then the assignment of a non-zero cost simulated action (e.g., a treatment, intervention, etc.) may lead to better simulated outcome.

As an example, in a clinical evaluation domain, the predictive risk score may an ASCVD score that predicts a likelihood of an adverse CVD event within the next 10 years. The general recommendation is that persons (of a certain age) with a risk of greater than 10% should be prescribed statins. The question of whether ASCVD accuracy may be improved by integrating with an expensive polygenic risk score may be theoretically answered through agent-based simulation once the building blocks of “actual risk” and PRS are synthetically sampled. Once the genotyped and non-genotyped population has been bifurcated, it is possible to monitor their outcomes through statistical sampling of “actual risk” on some discrete time steps provided it is known what the reduction in risk is from the intervention, which may generally be found in the documentation on clinical studies of the intervention. Collecting costs for the individual patients; including genotype costs, intervention costs, adverse risk event costs and potential costs of an adverse reaction to the intervention, it is possible to understand the cost-benefit trade-off of utilizing PRS and Whole genome sequencing.

FIG. 8 illustrates an example process 800 for explanatory purposes. Although the example process 800 depicts a particular sequence of steps/operations, the sequence may be altered without departing from the scope of the present disclosure. For example, some of the steps/operations depicted may be performed in parallel or in a different sequence that does not materially impact the function of the process 800. In other examples, different components of an example device or system that implements the process 800 may perform functions at substantially the same time or in a specific sequence.

According to some examples, the process 800 includes, at step/operation 804, generating a predictive risk score and/or a simulated risk score for an agent data object. For example, the computing system 100 may generate the predictive risk score and/or the simulated risk score for the agent data object in accordance with one or more techniques described herein.

According to some examples, the process 800 includes, at step/operation 806, determining a simulated action for the agent data object based on the predictive risk score. For example, the computing system 100 may determine the simulated action for the agent data object based on the predictive risk score. In some embodiments, the computing system 100 determines a first simulated action for the agent data object based on the predictive risk score. For example, the computing system 100 may determine the first simulated action in the event that the predictive risk score does not satisfy a risk threshold. The risk threshold may depend on the evaluation domain. In some examples, for instance in a clinical evaluation domain, the risk threshold may define an intervention threshold for an individual. A first simulated action may indicate that an intervention is not needed, whereas a second simulated action may indicate that the intervention is needed. In response to determining the first simulated action, the process 800 may proceed to step/operation 802 in which the first simulated action is assigned to the agent data object. In the event that the first simulated action is not determined for the agent data object based on the predictive risk score, the process may proceed to step/operation 808 to generate a refined risk score.

According to some examples, the process 800 includes, at step/operation 808, generating a refined risk score for the agent data object. For example, the computing system 100 may generate the refined risk score for the agent data object in accordance with one or more techniques described herein.

According to some examples, the process 800 includes, at step/operation 810, determining a simulated action for the agent data object based on the refined risk score. For example, the computing system 100 may determine the simulated action for the agent data object based on the refined risk score. In some embodiments, the computing system 100 determines a first simulated action for the agent data object in the event that the refined risk score does not satisfy the risk threshold. In response to determining the first simulated action, the process 800 may proceed to step/operation 802 in which the first simulated action is assigned to the agent data object. In some embodiments, the computing system 100 determines a second simulated action for the agent data object in the event that the refined risk score does satisfy the risk threshold. In the event that the second simulated action is determined for the agent data object based on the refined risk score, the process 800 may proceed to step/operation 812 in which the second simulated action is assigned to the agent data object.

In some embodiment, the first simulated action and the second simulated action identify two separate actions, such as an intervention or a non-intervention. After one or more iterations of the agent-based simulation, the computing system 100 may generate one or more return metrics that are based on the two separate actions. For example, the computing system 100 may determine a simulated outcome for the agent data object based on a simulated risk score for the agent data object, the first simulated action, and/or the second simulated action. The computing system 100 may update the agent dataset based on the simulated outcome, the first simulated action, and the second simulated action. After a plurality of iterations, the computing system 100 may analyze the results of each of the iterations to determine the return metrics.

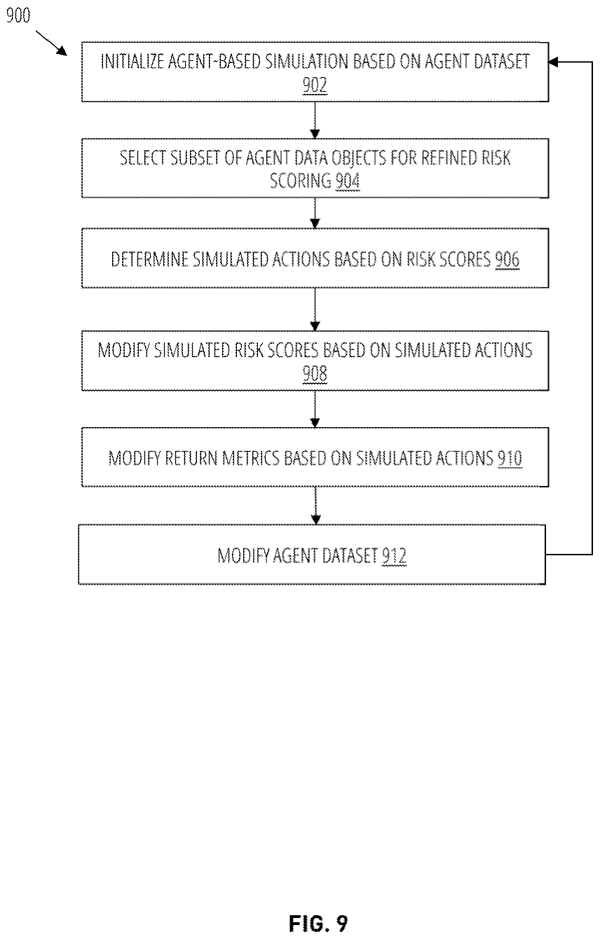

FIG. 9 is a flowchart showing an example of a process 900 for performing an agent-based simulation in accordance with some embodiments discussed herein. The process 900 leverages a plurality of different simulation, statistical, and data augmentation techniques to simulate the performance of a multiple different risk scoring techniques without relying on real world datasets specific to each of the targeted techniques. The process 900 may be implemented by one or more computing devices, entities, and/or systems described herein. For example, via the various steps/operations of the process 900, the computing system 100 may leverage the simulation, statistical, and data augmentation techniques to overcome various limitations with conventional simulation and risk scoring techniques that are traditionally limited by the deficiencies of real-world data.

FIG. 9 illustrates an example process 900 for explanatory purposes. Although the example process 900 depicts a particular sequence of steps/operations, the sequence may be altered without departing from the scope of the present disclosure. For example, some of the steps/operations depicted may be performed in parallel or in a different sequence that does not materially impact the function of the process 900. In other examples, different components of an example device or system that implements the process 900 may perform functions at substantially the same time or in a specific sequence.

According to some examples, the process 900 includes, at step/operation 902, initializing an agent-based simulation iteration based on an agent dataset. For example, the computing system 100 may initialize the agent-based simulation iteration based on the agent dataset. For instance, at the start of the agent-based simulation, a plurality of predictive risks (e.g., baseline ASCVD score, etc.), a plurality of simulated risks (e.g., actual risks), and/or a plurality of refined risks (e.g., PRS score, etc.) may be generated for an agent dataset. In some examples, in a clinical evaluation domain, each agent data object may be initialized as non-CVD before the first iteration of the agent-based simulation.

According to some examples, the process 900 includes, at step/operation 904, selecting a subset of agent data objects for refined risk scoring. For example, the computing system 100 may select the subset of agent data objects for refined risk scoring. The subset of the agent data objects may include agent data objects with predictive risk scores within a selection range. For example, in a clinical evaluation domain, agent data objects may be selected based on their ASCVD range. For example, five ranges may be used including experimental parameters, such as 5%-10%, 6%-10%, 7% to 10%, 8%-10% and/or 9%-10%,

According to some examples, the process 900 includes, at step/operation 906, determining simulated actions based on the risk scores. For example, the computing system 100 may determine the simulated actions based on the risk scores. By way of example, in a clinical evaluation domain, the simulated actions may be the prescription or non-prescription of statins to lower risk of CVD. If a non-sequenced agent data object has a ASCVD risk >10% statins may be prescribed. In addition, or alternatively, if a sequenced agent data object has ASCVD & PRS risk combined >10%, statins are prescribed. In some examples, if the sequenced agent data object has an ASCVD risk score >10% but an ASCVD+PRS risk score <10% statins are still prescribed as it is assumed that a member cannot be down-scored by the addition of PRS scores only up-scored.

According to some examples, the process 900 includes, at step/operation 908, modifying the simulated risk scores based on the simulated actions. For example, the computing system 100 may modify the simulated risk scores based on the simulated actions. By way of example, in a clinical domain, statins may reduce risk of initial CVD event by 25%. This may be realized by reducing simulated risk score for agent data objects that have been prescribed statins.

According to some examples, the process 900 includes, at step/operation 910, modifying the return metrics based on the simulated actions. For example, the computing system 100 may modify the return metrics based on the simulated actions. By way of example, in a clinical domain, the cost of statin over a year may be uniformly distributed between $200-$300 and sequencing cost may be assumed to be $500 per individual. The return metrics may be modified to reflect extra costs for agent data objects that have been prescribed statins. In some examples, the selected genotyped cohort may be cloned, and it is assumed they are not genotyped=>they have no additional PRS information. In this way, two sets of identical cohorts with/with PRS information may be compared to generate the return metrics. By way of example, the return metrics may describe at least one of: (i) a predictive risk score range (e.g., ASCVD range, etc.), (ii) a percentage of the agent dataset for which a refined risk score is generated (e.g., % of population genotyped, etc.), (iii) a percentage of the agent dataset for which the refined risk score is greater than the predictive risk score (e.g., % of population genotyped upscored), (iv) cost savings per agent data object (e.g., CVD costs saving per person, etc.), (v) net cost savings per agent data object, (vi) reduced adverse outcomes per agent data object (e.g., reduced CVD event per 100,000 persons, etc.), (vii) reduced severe adverse outcomes per agent data object (e.g., reduced CVD deaths per 100,000 persons, etc.).

According to some examples, the process 900 includes, at step/operation 912, modifying the agent dataset. For example, the computing system 100 may modify the agent dataset by churning one or more agent data objects based on the simulated passage of time and/or simulated outcomes. After modifying the agent dataset, the process 900 may return to step/operation 902 to perform a subsequent iteration of the agent-based simulation based on the modified agent dataset.

As one example, in a clinical domain, an agent data object may churn with a rate defined by both their age and perceived wellness. An agent data object prescribed with statins may adhere with some diminishing rate before becoming constant after 3 years. An agent data object may be removed from the simulation based on non-CVD related issues with rates based of the CDC mortality life tables. An agent data object may have their first CVD episode which is sampled from a respective simulated risk and updated every year to account for age. The cost of the year of first CVD episode and every subsequent year may be Lognormal ($13 k, $27 k). An agent data object may have a subsequent CVD episode, which may be determined based on another model for subsequent CVD events (e.g., as ASCVD may only be applicable to non-CVD lives, etc.). The determination may be based on several factors such as age, gender, vitals, ethnicity, time since last CVD event and underlying conditions. The cost for the year of subsequent CVD event and every year after is Lognormal ($63 k, $123 k), An agent data object may be removed from the agent dataset based on a CVD related issued which may be derived from a similar algorithm as subsequent CVD episodes. A small minority of agent data objects may have a minor or major adverse statin event with non-negligible probability and an associated cost.

After each iteration of the agent-based simulation, the agent dataset may be modified to simulate the passage of a year. The agent-based simulation may simulate any time range by performing one or more different numbers of iterations. In some embodiments, the agent-based simulation is performed for twenty iterations (e.g., representing 20 years). In some embodiments, the agent-based simulation is replicated ten times.

### VI. Conclusion

Many modifications and other embodiments will come to mind to one skilled in the art to which the present disclosure pertains having the benefit of the teachings presented in the foregoing descriptions and the associated drawings. Therefore, it is to be understood that the present disclosure is not to be limited to the specific embodiments disclosed and that modifications and other embodiments are intended to be included within the scope of the appended claims. Although specific terms are employed herein, they are used in a generic and descriptive sense only and not for purposes of limitation.

### VII. Examples

Example 1. A computer-implemented method, the computer-implemented method comprising: generating, by one or more processors and using a risk prediction model, a plurality of predictive risk scores for an agent dataset associated with an agent-based simulation; generating, by the one or more processors, a plurality of simulated risk scores for the agent dataset based on the plurality of predictive risk scores and a first performance metric corresponding to the risk prediction model; generating, by the one or more processors, a plurality of refined risk scores for the agent dataset based on the plurality of simulated risk scores and a second performance metric corresponding to a target risk refinement model; and generating, by the one or more processors, one or more return metrics for the target risk refinement model based on one or more iterations of the agent-based simulation, wherein an iteration of the agent-based simulation is performed using the plurality of predictive risk scores, the plurality of simulated risk scores, and the plurality of refined risk scores.

Example 2. The computer-implemented method of example 1 further comprising: generating the agent dataset based on a real-world dataset for a population of interest, wherein the agent dataset comprises a plurality of agent data objects corresponding to the population of interest, and wherein an agent data object of the plurality of agent data objects comprises a plurality of agent markers derived from the population of interest.

Example 3. The computer-implemented method of example 2, wherein generating the plurality of predictive risk scores for the agent dataset comprises: generating, using the risk prediction model, a predictive risk score for the agent data object based on the plurality of agent markers.

Example 4. The computer-implemented method of examples 2 of 3, wherein: (i) a predictive risk score for the agent data object represents a first predicted likelihood of an adverse outcome for the agent data object during the one or more iterations of the agent-based simulation, (ii) a simulated risk score for the agent data object represents an actual likelihood of the adverse outcome for the agent data object during the one or more iterations of the agent-based simulation, and (iii) a refined risk score for the agent data object represents a second predicted likelihood of the adverse outcome for the agent data object during the one or more iterations of the agent-based simulation.

Example 5. The computer-implemented method of any of the preceding examples, wherein the first performance metric comprises an area under the ROC curve (AUC) metric for the risk prediction model.

Example 6. The computer-implemented method of example 5, wherein generating the plurality of simulated risk scores for the agent dataset comprises: generating a plurality of score perturbations for the plurality of predictive risk scores; generating a first plurality of simulated risk scores by augmenting one or more of the plurality of predictive risk scores with the plurality of score perturbations; generating a plurality of simulated outcomes for the agent dataset based on the plurality of predictive risk scores; generating, using a performance evaluation model, a simulated performance metric for the risk prediction model based on the plurality of simulated outcomes; and generating the plurality of simulated risk scores based on a comparison between the simulated performance metric and the AUC metric.

Example 7. The computer-implemented method of any of the preceding examples, wherein the first performance metric comprises an r-squared (R2) metric for the risk prediction model.

Example 8. The computer-implemented method of example 7, wherein generating the plurality of simulated risk scores for the agent dataset comprises: generating an exponential distribution based on an average predictive risk score of the plurality of predictive risk scores; identifying a first plurality of simulated risk scores by sampling a plurality of distribution scores from the exponential distribution; and generating the plurality of simulated risk scores based on the plurality of predictive risk scores, the first plurality of simulated risk scores, and the R2 metric.

Example 9. The computer-implemented method of any of the preceding examples, wherein generating the plurality of refined risk scores for the agent dataset comprises: generating a Cohen's deviation based on a second predictive metric for the target risk refinement model; identifying one or more refined sample deviations from a standard normal distribution; identifying one or more offset refined sample deviations from an offset standard normal distribution that is offset by the Cohen's deviation; and generating the plurality of refined risk scores by randomly augmenting one or more of the plurality of predictive risk scores with at least one of the one or more refined sample deviations or the one or more offset refined sample deviations.

Example 10. The computer-implemented method of example 9, wherein generating the plurality of refined risk scores further comprises: generating a plurality of simulated outcomes for the agent dataset based on the plurality of simulated risk scores, wherein each of the plurality of simulated outcomes identify at least one of a positive outcome or an adverse outcome for an agent data object of the agent dataset; identifying an adverse subset of the plurality of agent data objects from the agent dataset based on the plurality of simulated outcomes, wherein each adverse agent data object of the adverse subset is associated with the adverse outcome, wherein the adverse subset of the plurality of agent data objects are associated with a first subset of the plurality of predictive risk scores; and randomly augmenting one or more of the first subset of the plurality of predictive risk scores with the one or more refined sample deviations.

Example 11. The computer-implemented method of example 10, wherein generating the plurality of refined risk scores further comprises: identifying a positive subset of the plurality of agent data objects from the agent dataset based on the plurality of simulated outcomes, wherein each positive agent data object of the positive subset is associated with the positive outcome, wherein the positive subset of the plurality of agent data objects are associated with a second subset of the plurality of predictive risk scores; and randomly augmenting one or more of the second subset of the plurality of predictive risk scores with the one or more offset refined sample deviations.

Example 12. The computer-implemented method of any of the preceding examples, wherein the iteration of the agent-based simulation comprises: determining a first simulated action for an agent data object of the agent dataset based on a predictive risk score for the agent data object; determining a second simulated action for the agent data object based on a refined risk score for the agent data object; determining a simulated outcome for the agent data object based on a simulated risk score for the agent data object, the first simulated action, and the second simulated action; and updating the agent dataset based on the simulated outcome, the first simulated action, and the second simulated action.

Example 13. The computer-implemented method of example 12, wherein the first simulated action and the second simulated action identify two separate actions, and wherein the one or more return metrics are based on the two separate actions.

Example 14. The computer-implemented method of any of the preceding examples, wherein the one or more return metrics are representative of a real-world benefit of using the target risk refinement model.

Example 15. A computing apparatus comprising memory and one or more processors communicatively coupled to the memory, the one or more processors configured to: generate, using a risk prediction model, a plurality of predictive risk scores for an agent dataset associated with an agent-based simulation; generate a plurality of simulated risk scores for the agent dataset based on the plurality of predictive risk scores and a first performance metric corresponding to the risk prediction model; generate a plurality of refined risk scores for the agent dataset based on the plurality of simulated risk scores and a second performance metric corresponding to a target risk refinement model; and generate one or more return metrics for the target risk refinement model based on one or more iterations of the agent-based simulation, wherein an iteration of the agent-based simulation is performed using the plurality of predictive risk scores, the plurality of simulated risk scores, and the plurality of refined risk scores.

Example 16. The computing apparatus of example 15, wherein the one or more processors are further configured to: generate the agent dataset based on a real-world dataset for a population of interest, wherein the agent dataset comprises a plurality of agent data objects corresponding to the population of interest, and wherein an agent data object of the plurality of agent data objects comprises a plurality of agent markers derived from the population of interest.

Example 17. The computing apparatus of example 16, wherein the one or more processors are further configured to: generate, using the risk prediction model, a predictive risk score for the agent data object based on the plurality of agent markers.

Example 18. One or more non-transitory computer-readable storage media including instructions that, when executed by one or more processors, cause the one or more processors to: generate, using a risk prediction model, a plurality of predictive risk scores for an agent dataset associated with an agent-based simulation; generate a plurality of simulated risk scores for the agent dataset based on the plurality of predictive risk scores and a first performance metric corresponding to the risk prediction model; generate a plurality of refined risk scores for the agent dataset based on the plurality of simulated risk scores and a second performance metric corresponding to a target risk refinement model; and generate one or more return metrics for the target risk refinement model based on one or more iterations of the agent-based simulation, wherein an iteration of the agent-based simulation is performed using the plurality of predictive risk scores, the plurality of simulated risk scores, and the plurality of refined risk scores.

Example 19. The one or more non-transitory computer-readable storage media of example 18, wherein: (i) a predictive risk score for an agent data object of the agent dataset represents a first predicted likelihood of an adverse outcome for the agent data object during the one or more iterations of the agent-based simulation, (ii) a simulated risk score for the agent data object represents an actual likelihood of the adverse outcome for the agent data object during the one or more iterations of the agent-based simulation, and (iii) a refined risk score for the agent data object represents a second predicted likelihood of the adverse outcome for the agent data object during the one or more iterations of the agent-based simulation.

Example. 20. The one or more non-transitory computer-readable storage media of examples 18 or 19, wherein the first performance metric comprises an area under the ROC curve (AUC) metric or an r-squared (R2) metric for the risk prediction model.

1. A computer-implemented method, the computer-implemented method comprising:
generating, by one or more processors and using a risk prediction model, a plurality of predictive risk scores for an agent dataset associated with an agent-based simulation;
generating, by the one or more processors, a plurality of simulated risk scores for the agent dataset based on the plurality of predictive risk scores and a first performance metric corresponding to the risk prediction model;
generating, by the one or more processors, a plurality of refined risk scores for the agent dataset based on the plurality of simulated risk scores and a second performance metric corresponding to a target risk refinement model; and
generating, by the one or more processors, one or more return metrics for the target risk refinement model based on one or more iterations of the agent-based simulation, wherein an iteration of the agent-based simulation is performed using the plurality of predictive risk scores, the plurality of simulated risk scores, and the plurality of refined risk scores.

2. The computer-implemented method of claim 1 further comprising:
generating the agent dataset based on a real-world dataset for a population of interest, wherein the agent dataset comprises a plurality of agent data objects corresponding to the population of interest, and wherein an agent data object of the plurality of agent data objects comprises a plurality of agent markers derived from the population of interest.

3. The computer-implemented method of claim 2, wherein generating the plurality of predictive risk scores for the agent dataset comprises:
generating, using the risk prediction model, a predictive risk score for the agent data object based on the plurality of agent markers.

4. The computer-implemented method of claim 2, wherein:
(i) a predictive risk score for the agent data object represents a first predicted likelihood of an adverse outcome for the agent data object during the one or more iterations of the agent-based simulation,
(ii) a simulated risk score for the agent data object represents an actual likelihood of the adverse outcome for the agent data object during the one or more iterations of the agent-based simulation, and
(iii) a refined risk score for the agent data object represents a second predicted likelihood of the adverse outcome for the agent data object during the one or more iterations of the agent-based simulation.

5. The computer-implemented method of claim 1, wherein the first performance metric comprises an area under the ROC curve (AUC) metric for the risk prediction model.

6. The computer-implemented method of claim 5, wherein generating the plurality of simulated risk scores for the agent dataset comprises:
generating a plurality of score perturbations for the plurality of predictive risk scores;
generating a first plurality of simulated risk scores by augmenting one or more of the plurality of predictive risk scores with the plurality of score perturbations;
generating a plurality of simulated outcomes for the agent dataset based on the plurality of predictive risk scores;
generating, using a performance evaluation model, a simulated performance metric for the risk prediction model based on the plurality of simulated outcomes; and
generating the plurality of simulated risk scores based on a comparison between the simulated performance metric and the AUC metric.

7. The computer-implemented method of claim 1, wherein the first performance metric comprises an r-squared (R2) metric for the risk prediction model.

8. The computer-implemented method of claim 7, wherein generating the plurality of simulated risk scores for the agent dataset comprises:
generating an exponential distribution based on an average predictive risk score of the plurality of predictive risk scores;
identifying a first plurality of simulated risk scores by sampling a plurality of distribution scores from the exponential distribution; and
generating the plurality of simulated risk scores based on the plurality of predictive risk scores, the first plurality of simulated risk scores, and the R2 metric.

9. The computer-implemented method of claim 1, wherein generating the plurality of refined risk scores for the agent dataset comprises:
generating a Cohen's deviation based on a second predictive metric for the target risk refinement model;
identifying one or more refined sample deviations from a standard normal distribution;
identifying one or more offset refined sample deviations from an offset standard normal distribution that is offset by the Cohen's deviation; and
generating the plurality of refined risk scores by randomly augmenting one or more of the plurality of predictive risk scores with at least one of the one or more refined sample deviations or the one or more offset refined sample deviations.

10. The computer-implemented method of claim 9, wherein generating the plurality of refined risk scores further comprises:
generating a plurality of simulated outcomes for the agent dataset based on the plurality of simulated risk scores, wherein each of the plurality of simulated outcomes identify at least one of a positive outcome or an adverse outcome for an agent data object of the agent dataset;
identifying an adverse subset of the plurality of agent data objects from the agent dataset based on the plurality of simulated outcomes, wherein each adverse agent data object of the adverse subset is associated with the adverse outcome, wherein the adverse subset of the plurality of agent data objects are associated with a first subset of the plurality of predictive risk scores; and
randomly augmenting one or more of the first subset of the plurality of predictive risk scores with the one or more refined sample deviations.

11. The computer-implemented method of claim 10, wherein generating the plurality of refined risk scores further comprises:
identifying a positive subset of the plurality of agent data objects from the agent dataset based on the plurality of simulated outcomes, wherein each positive agent data object of the positive subset is associated with the positive outcome, wherein the positive subset of the plurality of agent data objects are associated with a second subset of the plurality of predictive risk scores; and
randomly augmenting one or more of the second subset of the plurality of predictive risk scores with the one or more offset refined sample deviations.

12. The computer-implemented method of claim 1, wherein the iteration of the agent-based simulation comprises:
determining a first simulated action for an agent data object of the agent dataset based on a predictive risk score for the agent data object;
determining a second simulated action for the agent data object based on a refined risk score for the agent data object;
determining a simulated outcome for the agent data object based on a simulated risk score for the agent data object, the first simulated action, and the second simulated action; and
updating the agent dataset based on the simulated outcome, the first simulated action, and the second simulated action.

13. The computer-implemented method of claim 12, wherein the first simulated action and the second simulated action identify two separate actions, and wherein the one or more return metrics are based on the two separate actions.

14. The computer-implemented method of claim 1, wherein the one or more return metrics are representative of a real-world benefit of using the target risk refinement model.

15. A computing apparatus comprising memory and one or more processors communicatively coupled to the memory, the one or more processors configured to:
generate, using a risk prediction model, a plurality of predictive risk scores for an agent dataset associated with an agent-based simulation;
generate a plurality of simulated risk scores for the agent dataset based on the plurality of predictive risk scores and a first performance metric corresponding to the risk prediction model;
generate a plurality of refined risk scores for the agent dataset based on the plurality of simulated risk scores and a second performance metric corresponding to a target risk refinement model; and
generate one or more return metrics for the target risk refinement model based on one or more iterations of the agent-based simulation, wherein an iteration of the agent-based simulation is performed using the plurality of predictive risk scores, the plurality of simulated risk scores, and the plurality of refined risk scores.

16. The computing apparatus of claim 15, wherein the one or more processors are further configured to:
generate the agent dataset based on a real-world dataset for a population of interest, wherein the agent dataset comprises a plurality of agent data objects corresponding to the population of interest, and wherein an agent data object of the plurality of agent data objects comprises a plurality of agent markers derived from the population of interest.

17. The computing apparatus of claim 16, wherein the one or more processors are further configured to:
generate, using the risk prediction model, a predictive risk score for the agent data object based on the plurality of agent markers.

18. One or more non-transitory computer-readable storage media including instructions that, when executed by one or more processors, cause the one or more processors to:
generate, using a risk prediction model, a plurality of predictive risk scores for an agent dataset associated with an agent-based simulation;
generate a plurality of simulated risk scores for the agent dataset based on the plurality of predictive risk scores and a first performance metric corresponding to the risk prediction model;
generate a plurality of refined risk scores for the agent dataset based on the plurality of simulated risk scores and a second performance metric corresponding to a target risk refinement model; and
generate one or more return metrics for the target risk refinement model based on one or more iterations of the agent-based simulation, wherein an iteration of the agent-based simulation is performed using the plurality of predictive risk scores, the plurality of simulated risk scores, and the plurality of refined risk scores.

19. The one or more non-transitory computer-readable storage media of claim 18, wherein:
(i) a predictive risk score for an agent data object of the agent dataset represents a first predicted likelihood of an adverse outcome for the agent data object during the one or more iterations of the agent-based simulation,
(ii) a simulated risk score for the agent data object represents an actual likelihood of the adverse outcome for the agent data object during the one or more iterations of the agent-based simulation, and
(iii) a refined risk score for the agent data object represents a second predicted likelihood of the adverse outcome for the agent data object during the one or more iterations of the agent-based simulation.

20. The one or more non-transitory computer-readable storage media of claim 18, wherein the first performance metric comprises an area under the ROC curve (AUC) metric or an r-squared (R2) metric for the risk prediction model.
